SEQUENCE LISTING (36 sequences)

SEQ ID NO: 1 - protein, 50 aa, Artificial Sequence
  Synthetic construct
MNPNQKITTIGSICLVVGLISLILQIGNIISIWISHSIQTGSQNHTGICN

SEQ ID NO: 2 - protein, 80 aa, Artificial Sequence
  Synthetic construct
MNPNQKITTIGSICLVVGLISLILQIGNIISIWISHSIQTGSQNHTGICNQNIITYKNSTWVKDTTSVILTGNSSLCPIR

SEQ ID NO: 3 - protein, 100 aa, Artificial Sequence
  Synthetic construct
MNPNQKITTIGSICLVVGLISLILQIGNIISIWISHSIQTGSQNHTGICNQNIITYKNSTWVKDTTSVILTGNSSLCPIRGWAIYSKDNSIRIGSKGDVF

SEQ ID NO: 4 - protein, 120 aa, Artificial Sequence
  Synthetic construct
MNPNQKITTIGSICLVVGLISLILQIGNIISIWISHSIQTGSQNHTGICNQNIITYKNSTWVKDTTSVILTGNSSLCPIRGWAIYSKDNSIRIGSKGDVFVIREPFISCSHLECRTFFLT

SEQ ID NO: 5 - protein, 6 aa, Artificial Sequence
  Synthetic construct
LEGGGS

SEQ ID NO: 6 - protein, 4 aa, Artificial Sequence
  Synthetic construct
TGSG

SEQ ID NO: 7 - protein, 8 aa, Artificial Sequence
  Synthetic construct
GGSGGGSG

SEQ ID NO: 8 - protein, 5 aa, Artificial Sequence
  Synthetic construct
GGGGS

SEQ ID NO: 9 - protein, 4 aa, Artificial Sequence
  Synthetic construct
GGGS

SEQ ID NO: 10 - protein, 6 aa, Artificial Sequence
  Synthetic construct
GSGGGG

SEQ ID NO: 11 - protein, 6 aa, Artificial Sequence
  Synthetic construct
GLYGLY

SEQ ID NO: 12 - protein, 5 aa, Artificial Sequence
  Synthetic construct
EAAAK

SEQ ID NO: 13 - protein, 7 aa, Artificial Sequence
  Synthetic construct
AEAAAKA

SEQ ID NO: 14 - protein, 5 aa, Artificial Sequence
  Synthetic construct
PAPAP

SEQ ID NO: 15 - protein, 12 aa, Artificial Sequence
  Synthetic construct
APAPAPAPAPAP

SEQ ID NO: 16 - protein, 279 aa, Artificial Sequence
  Synthetic construct
MNPNQKITTIGSICLVVGLISLILQIGNIISIWISHSIQTGSQNHTGICNRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 309 aa, Artificial Sequence
  Synthetic construct
MNPNQKITTIGSICLVVGLISLILQIGNIISIWISHSIQTGSQNHTGICNQNIITYKNSTWVKDTTSVILTGNSSLCPIRRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 329 aa, Artificial Sequence
  Synthetic construct
MNPNQKITTIGSICLVVGLISLILQIGNIISIWISHSIQTGSQNHTGICNQNIITYKNSTWVKDTTSVILTGNSSLCPIRGWAIYSKDNSIRIGSKGDVFRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 349 aa, Artificial Sequence
  Synthetic construct
MNPNQKITTIGSICLVVGLISLILQIGNIISIWISHSIQTGSQNHTGICNQNIITYKNSTWVKDTTSVILTGNSSLCPIRGWAIYSKDNSIRIGSKGDVFVIREPFISCSHLECRTFFLTRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 15 aa, Artificial Sequence
  Synthetic construct
DKTHTCPPCPAPELL

SEQ ID NO: 21 - protein, 11 aa, Artificial Sequence
  Synthetic construct
TCPPCPAPELL

SEQ ID NO: 22 - protein, 212 aa, Artificial Sequence
  Synthetic construct
GGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 105 aa, Artificial Sequence
  Synthetic construct
GGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 24 - protein, 107 aa, Artificial Sequence
  Synthetic construct
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 347 aa, Artificial Sequence
  Synthetic construct
MNPNQKITTIGSICLVVGLISLILQIGNIISIWISHSIQTGSQNHTGICNQNIITYKNSTWVKDTTSVILTGNSSLCPIRGWAIYSKDNSIRIGSKGDVFVIREPFISCSHLECRTFFLTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 211 aa, Artificial Sequence
  Synthetic construct
GGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 27 - protein, 105 aa, Artificial Sequence
  Synthetic construct
GGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAK

SEQ ID NO: 28 - protein, 347 aa, Artificial Sequence
  Synthetic construct
MNPNQKITTIGSICLVVGLISLILQIGNIISIWISHSIQTGSQNHTGICNQNIITYKNSTWVKDTTSVILTGNSSLCPIRGWAIYSKDNSIRIGSKGDVFVIREPFISCSHLECRTFFLTDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 347 aa, Artificial Sequence; identical to SEQ ID NO: 25
  Synthetic construct

SEQ ID NO: 30 - protein, 347 aa, Artificial Sequence; identical to SEQ ID NO: 25
  Synthetic construct

SEQ ID NO: 31 - protein, 559 aa, Artificial Sequence
  Synthetic construct
MNPNQKITTIGSICLVVGLISLILQIGNIISIWISHSIQTGSQNHTGICNQNIITYKNSTWVKDTTSVILTGNSSLCPIRGWAIYSKDNSIRIGSKGDVFVIREPFISCSHLECRTFFLTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - DNA, 870 bp, Artificial Sequence
  Synthetic construct
gaattccagggggtttaaaatgaatccaaatcagaaaataacaaccattggatcaatctgtctggtagtcggactaattagcctaatattgcaaatagggaatataatctcaatatggattagccattcaattcaaactggaagtcaaaaccatactggaatatgcaacagatctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaatgagtgctagctgg

SEQ ID NO: 33 - DNA, 960 bp, Artificial Sequence
  Synthetic construct
gaattccagggggtttaaaatgaatccaaatcagaaaataacaaccattggatcaatctgtctggtagtcggactaattagcctaatattgcaaatagggaatataatctcaatatggattagccattcaattcaaactggaagtcaaaaccatactggaatatgcaaccaaaacatcattacctataaaaatagcacctgggtaaaggacacaacttcagtgatattaaccggcaattcatctctttgtcccatccgtagatctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaatgagtgctagctgg

SEQ ID NO: 34 - DNA, 1020 bp, Artificial Sequence
  Synthetic construct
gaattccagggggtttaaaatgaatccaaatcagaaaataacaaccattggatcaatctgtctggtagtcggactaattagcctaatattgcaaatagggaatataatctcaatatggattagccattcaattcaaactggaagtcaaaaccatactggaatatgcaaccaaaacatcattacctataaaaatagcacctgggtaaaggacacaacttcagtgatattaaccggcaattcatctctttgtcccatccgtgggtgggctatatacagcaaagacaatagcataagaattggttccaaaggagacgtttttagatctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaatgagtgctagctgg

SEQ ID NO: 35 - DNA, 1080 bp, Artificial Sequence
  Synthetic construct
gaattccagggggtttaaaatgaatccaaatcagaaaataacaaccattggatcaatctgtctggtagtcggactaattagcctaatattgcaaatagggaatataatctcaatatggattagccattcaattcaaactggaagtcaaaaccatactggaatatgcaaccaaaacatcattacctataaaaatagcacctgggtaaaggacacaacttcagtgatattaaccggcaattcatctctttgtcccatccgtgggtgggctatatacagcaaagacaatagcataagaattggttccaaaggagacgtttttgtcataagagagccctttatttcatgttctcacttggaatgcaggaccttttttctgaccagatctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaatgagtgctagctgg

SEQ ID NO: 36 - protein, 227 aa, Artificial Sequence
  Synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2024/0010976 A1
(43) Pub. Date: Jan. 11, 2024

(54) METHODS AND COMPOSITIONS FOR STIMULATING GAMMA DELTA T CELLS

(71) Applicant: UNIVERSITY OF CENTRAL FLORIDA RESEARCH FOUNDATION, INC., Orlando, FL (US)

(72) Inventors: Alicja COPIK, Orlando, FL (US); Jeremiah OYER, Orlando, FL (US); Thomas DIEFFENTHALLER, Orlando, FL (US); Griff PARKS, Orlando, FL (US)

(21) Appl. No.: 18/021,144

(22) PCT Filed: Aug. 12, 2021
(86) PCT No.: PCT/US2021/045643

§ 371 Date: Feb. 13, 2023

Related U.S. Application Data
(60) Provisional application No. 63/064,832, filed on Aug. 12, 2020.

(51) Int. Cl. C12N 5/0783; A61K 35/17; A61K 45/06

(52) U.S. Cl. CPC C12N 5/0636; A61K 35/17; A61K 45/06; C12N 2501/73; C12N 2502/30; C12N 2501/599

(57) ABSTRACT

Compositions and methods for stimulating γδ T cell expansion and cytotoxicity are described. Therapeutic compositions and methods using expanded and stimulated γδ T cells are described.

### CROSS REFERENCE TO RELATED APPLICATIONS

This application is a United States National Phase Patent Application of International Patent Application Number PCT/US2021/045643, filed Aug. 12, 2021, which claims the benefit of priority to U.S. Provisional Application No. 63/064,832, filed Aug. 12, 2020, contents of which are incorporated by reference herein in their entireties.

### SEQUENCE LISTING

A Sequence Listing was filed in electronic format on Feb. 6, 2023. The Sequence Listing was provided as a file entitled “10613_078US1_2023_02_13_ST25.txt”, created Feb. 13, 2023, which is 48,933 bytes in size. The information in the electronic format of the Sequence Listing is incorporated herein by reference in its entirety.

### BACKGROUND

Cellular therapy that utilizes innate effector populations such as γδ T cells provides a promising treatment platform for various illnesses. Since these immune cells are present in blood in limited quantities application of cellular therapy requires efficient methods of cell expansion to enable generation of cell quantities sufficient to produce therapeutic dosages. Challenges to fully realizing the clinical potential of γδ T cell therapy include obtaining large numbers of robust, healthy γδ T cells that exhibit high cytotoxicity; ability to target the γδ T cell to a disease target; and, once introduced to a patient, having the γδ T cell sufficiently persist in vivo to achieve a therapeutic effect. What is needed are compositions and methods of expanding γδ T cells and uses thereof for treatment of diseases.

### SUMMARY

The invention here conceived of encompasses compositions and uses thereof for expanding γδ T cells that includes Fc domain of an antibody, that is competent for agonizing a Fc receptor (e.g., CD16), bound to a feeder cells, engineered particles, exosomes, or on some other solid support. The feeder cells, engineered particles, exosomes and other solid supports with bound Fc domain can also comprise one or more additional γδ T cell effector agent(s) such as membrane bound IL-21, 4-1BBL, other cytokines, adhesion molecules, and/or γδ T cell activating agents that simultaneously engage other stimulatory (or possibly inhibitory) receptors and corresponding signaling pathways. Engagement of the Fc receptor (e.g., CD16) by the aforementioned agents leads to expansion of an initial population of γδ T cells wherein the cells generated through expansion have a higher cytotoxicity than the initial population of cells. Additionally, this method can lead to co-expansion of γδ T and NK cells if NK cells are not removed prior to expansion. The combination of these two populations can lead to broader antitumor function and thus better efficacy. Such γδ T cells or γδ T/NK cell mixture can be utilized as therapeutics for treatment of diseases.

In some aspects, disclosed herein is a method for inducing, activating, and/or expanding of γδ T cells, comprising contacting at least one γδ T cell with an engineered feeder cell, an engineered plasma membrane particle, an exosome, or a solid support comprising a Fc domain bound to the external surface thereof through a transmembrane domain. The transmembrane domain may be a transmembrane domain of neuraminidase, a signal-anchor sequence from parainfluenza virus hemagglutinin-neuraminidase, a signal-anchor sequence from the transferrin receptor, a signal-anchor sequence from the MHC class II invariant chain, a signal-anchor sequence from P glycoprotein, a signal-anchor sequence from asialoglycoprotein receptor, and a signal-anchor sequence from a neutral endopeptidase. In some embodiments, the transmembrane domain comprises a parainfluenza virus hemagglutinin-neuraminidase (NA) peptide sequence comprising a sequence at least 81% identical to SEQ ID NO: 1. The transmembrane domain and the Fc domain may be linked via a peptide linker.

In some embodiments, the Fc domain comprises an immunoglobulin Fc domain selected from IgG1, IgG2, IgG3, IgG4, IgA and IgE. In some embodiment, the Fc domain binds to CD16.

The feeder cell may be a peripheral blood mononuclear cell (PBMC), a fibroblast, an epithelial cell, an endothelial cell, an antigen-presenting cell, or microbial cell, or a cell line, wherein the cell line may be RPMI8866, HFWT, 721.221, K562, or EBV-LCL.

In some embodiments, the method of any preceding aspect further comprises contacting the at least one γδ T cell with at least one γδ T cell effector agent, wherein the at least one γδ T cell effector agent is expressed on or bound to the external surface of the engineered feeder cell (i.e., membrane bound (mb)), the engineered plasma membrane particle, the exosome, or the solid support. The at least one γδ T cell effector agent may be a cytokine, an adhesion molecule, or a γδ T cell activating agent. In some embodiments, the at least one γδ T cell effector agent comprises 4-1BBL; CD80; CD86; MICA; UBLP; 2B4; LFA-1; agonist (e.g., agnositic antibody) or ligand for NKG2D, NKp46, NKp44, NKp30, or DNAM-1; agonist (e.g., agnositic antibody) or ligand for Notch, BCM/SLAMF2 or TLR; IL-2; IL-12; IL-18; IL-15; or IL-21; or any combination thereof. In some embodiments, the at least one γδ T cell effector agent comprises 4-1BBL, IL-18, IL-15, or IL-21, or any combination thereof (such as, for example, 4-1BBL and IL-21; 4-1BBL and IL-15; 4-1BBL and IL-18; 4-1BBL, IL-15, and IL-21; 4-1BBL, IL-18, and IL-21; 4-1BBL, IL-15, and IL-18; or 4-1BBL, IL-15, IL-18, and IL-21), including, but not limited to membrane bound 4-1BBL, IL-18, IL-15, or IL-21 or combinations thereof (such as, for example, mb4-1BBL and mbIL-21; mb4-1BBL and mbIL-15; mb4-1BBL and mbIL-18; mb4-1BBL, mbIL-15, and mbIL-21; mb4-1BBL, mbIL-18, and mbIL-21; mb4-1BBL, mbIL-15, and mbIL-18; or mb4-1BBL, mbIL-15, mbIL-18, and mbIL-21) as well as combinations of membrane bound and non-bound effector agents.

In some embodiments, the method of any preceding aspect comprises contacting the at least one γδ T cell with the feeder cell, the engineered particle, the exosome, or the solid support in vitro, in vivo, or ex vivo. In some embodiments, the expanded γδ T cells comprise Vδ2 subtype and/or Vδ1 subtype. The γδ T cells may be autologous, haploidentical, or allogeneic γδ T cells. In some embodiments, the γδ T cells are expanded for at least 14 days, wherein at least about 5%, 10%, 20%, 30%, 40%, 50%, or 60% of the cells in the expanded cells are γδ T-cells of the Vδ2 subtype.

In some embodiments, the γδ T cells expand at a faster rate over 14 days than a control γδ T cell population.

The γδ T cells expanded according to the methods of any preceding aspect can be an isolated cell population or in a mixed cell population. the mixed cell population can be depleted of NK cells prior to, during, or after expansion of the γδ T cells.

In some aspects, disclosed herein is a method of treating, decreasing, inhibiting, reducing, ameliorating, and/or preventing a cancer, metastasis, or an infectious disease in a subject comprising administering to the subject a therapeutically effective amount of γδ T cells expanded, activated, or induced according to the method of any preceding aspect.

In some aspects, disclosed herein is a method of treating, decreasing, inhibiting, reducing, ameliorating, and/or preventing a cancer, metastasis, or an infectious disease in a subject comprising
a. obtaining at least one γδ T cell;
b. contacting the least one γδ T cell with an engineered feeder cell, an engineered plasma membrane particle, an exosome, or a solid support comprising a Fc domain bound to the external surface thereof;
c. administering to the subject a therapeutically effective amount of the contacted γδ T cells to the subject.

In some embodiments, step b further comprises inducing, activating, and/or expanding the at least one γδ T cell following the contact with the engineered feeder cell, the engineered plasma membrane particle, the exosome, or the solid support comprising a Fc domain bound to the external surface thereof, wherein the γδ T cells are induced, activated, and/or expanded for at least 14 days.

In some embodiments, the engineered feeder cell, engineered plasma membrane particle, the exosome, or the solid support may further comprise at least one γδ T cell effector agent, wherein the at least one γδ T cell effector agent comprises 4-1BBL; CD80; CD86; MICA; UBLP; 2B4; LFA-1; agonist (e.g., agnositic antibody) or ligand for NKG2D, NKp46, NKp44, NKp30, or DNAM-1; agonist (e.g., agnositic antibody) or ligand for Notch, BCM/SLAMF2 or TLR; IL-2; IL-12; IL-18; IL-15; or IL-21; or any combination thereof. In some embodiments, the at least one γδ T cell effector agent comprises 4-1BBL, IL-18, IL-15, or IL-21, or any combination thereof (such as, for example, 4-1BBL and IL-21; 4-1BBL and IL-15; 4-1BBL and IL-18; 4-1BBL, IL-15, and IL-21; 4-1BBL, IL-18, and IL-21; 4-1BBL, IL-15, and IL-18; or 4-1BBL, IL-15, IL-18, and IL-21), including, but not limited to membrane bound 4-1BBL, IL-18, IL-15, or IL-21 or combinations thereof (such as, for example, mb4-1BBL and mbIL-21; mb4-1BBL and mbIL-15; mb4-1BBL and mbIL-18; mb4-1BBL, mbIL-15, and mbIL-21; mb4-1BBL, mbIL-18, and mbIL-21; mb4-1BBL, mbIL-15, and mbIL-18; or mb4-1BBL, mbIL-15, mbIL-18, and mbIL-21) as well as combinations of membrane bound and non-bound effector agents.

In some aspects, disclosed herein is a method of treating, decreasing, inhibiting, reducing, ameliorating, and/or preventing a cancer, metastasis, or an infectious disease in a subject by expanding, inducing, and/or activating endogenous γδ T cells in the subject, said method comprising administering to the subject an engineered plasma membrane particle, an exosome, or a solid support comprising a Fc domain bound to the external surface thereof, wherein the engineered feeder cell, engineered plasma membrane particle, the exosome, or the solid support may further comprise at least one γδ T cell effector agent, wherein the at least one γδ T cell effector agent comprises 4-1BBL; CD80; CD86; MICA; UBLP; 2B4; LFA-1; agonist (e.g., agnositic antibody) or ligand for NKG2D, NKp46, NKp44, NKp30, or DNAM-1; agonist (e.g., agnositic antibody) or ligand for Notch, BCM/SLAMF2 or TLR; IL-2; IL-12; IL-18; IL-15; or IL-21; or any combination thereof. In some embodiments, the at least one γδ T cell effector agent comprises 4-1BBL, IL-18, IL-15, or IL-21, or any combination thereof (such as, for example, 4-1BBL and IL-21; 4-1BBL and IL-15; 4-1BBL and IL-18; 4-1BBL, IL-15, and IL-21; 4-1BBL, IL-18, and IL-21; 4-1BBL, IL-15, and IL-18; or 4-1BBL, IL-15, IL-18, and IL-21), including, but not limited to membrane bound 4-1BBL, IL-18, IL-15, or IL-21 or combinations thereof (such as, for example, mb4-1BBL and mbIL-21; mb4-1BBL and mbIL-15; mb4-1BBL and mbIL-18; mb4-1BBL, mbIL-15, and mbIL-21; mb4-1BBL, mbIL-18, and mbIL-21; mb4-1BBL, mbIL-15, and mbIL-18; or mb4-1BBL, mbIL-15, mbIL-18, and mbIL-21) as well as combinations of membrane bound and non-bound effector agents.

In some embodiments, the methods of any preceding aspect further comprising administering to the subject an ex vivo composition comprising a fusion protein comprising a transmembrane domain linked to the amino terminus of an Fc domain and bound to an engineered feeder cell, an engineered plasma membrane particle, an exosome, or a solid support, in contact with an isolated mixed cell population comprising at least one γδ T cells comprising CD16 or a functional fragment thereof. In some embodiments, the ex vivo composition further comprises at least one γδ T cell effector agent, wherein the at least one γδ T cell effector agent comprises 4-1BBL; CD80; CD86; MICA; UBLP; 2B4; LFA-1; agonist (e.g., agnositic antibody) or ligand for NKG2D, NKp46, NKp44, NKp30, or DNAM-1; agonist (e.g., agnositic antibody) or ligand for Notch, BCM/SLAMF2 or TLR; IL-2; IL-12; IL-18; IL-15; or IL-21; or any combination thereof. In some embodiments, the at least one γδ T cell effector agent comprises 4-1BBL, IL-18, IL-15, or IL-21, or any combination thereof (such as, for example, 4-1BBL and IL-21; 4-1BBL and IL-15; 4-1BBL and IL-18; 4-1BBL, IL-15, and IL-21; 4-1BBL, IL-18, and IL-21; 4-1BBL, IL-15, and IL-18; or 4-1BBL, IL-15, IL-18, and IL-21), including, but not limited to membrane bound 4-1BBL, IL-18, IL-15, or IL-21 or combinations thereof (such as, for example, mb4-1BBL and mbIL-21; mb4-1BBL and mbIL-15; mb4-1BBL and mbIL-18; mb4-1BBL, mbIL-15, and mbIL-21; mb4-1BBL, mbIL-18, and mbIL-21; mb4-1BBL, mbIL-15, and mbIL-18; or mb4-1BBL, mbIL-15, mbIL-18, and mbIL-21) as well as combinations of membrane bound and non-bound effector agents. The engineered plasma membrane particle can comprise a plasma membrane and a plurality of microparticles or support surfaces, wherein the plasma membrane coats the plurality of microparticles or support surfaces. In some embodiments, the plurality of microparticles or surfaces comprise at least one of magnetic microparticles, silica beads, polystyrene beads, latex beads, micro-structures, a contrast agent, and a cancer therapeutic agent.

The methods disclosed herein are for treating a cancer, wherein the cancer is selected from the group consisting of a hematologic cancer, lymphoma, colorectal cancer, colon cancer, lung cancer, a head and neck cancer, ovarian cancer, prostate cancer, testicular cancer, renal cancer, skin cancer, cervical cancer, pancreatic cancer, and breast cancer. In one aspect, the cancer comprises a solid tumor. In another aspect, the cancer is selected from acute myeloid leukemia, myelodysplastic syndrome, chronic myeloid leukemia, acute lymphoblastic leukemia, myelofibrosis, multiple myeloma. In another aspect, the cancer is selected from a leukemia, a lymphoma, a sarcoma, a carcinoma and may originate in the marrow, brain, lung, breast, pancreas, liver, head and neck, skin, reproductive tract, prostate, colon, liver, kidney, intraperitoneum, bone, joint, eye.

In some embodiments, the method of any preceding aspect further comprises administering to the subject at least one cancer therapeutic agent in combination with the composition, wherein the at least one cancer therapeutic agent is selected from the group consisting of Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cab azitaxel, Cabometyx (Cabozantinib-S-Malate), Cabozantinib-S-Malate, CAF, Campath (Alemtuzumab), Camptosar, (Irinotecan Hydrochloride), Capecitabine, CAPDX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). In some embodiments, the at least one cancer therapeutic agent is selected from a chemotherapy agent (eg. CHOP, FLAG, 7+3), a drug based preparative regimen, or a combination thereof. (Cy-Flu, Bu-Flu, Flu-Mel).

In some aspects, the engineered particle further comprises one or more γδ T effector agents. In some aspects, an engineered particle further comprises at least one γδ T cell effector agent, wherein the γδ T cell effector agent is IL-21. In another aspect, the engineered particle further comprises at least two γδ T cell effector agents, wherein one of the at least two γδ T cell effector agents is IL-2.

In some aspects, the methods disclosed herein are for treating an infectious disease caused by a viral infection, wherein the viral infection comprises an infection of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papillomavirus, Respiratory syncytial virus, Adenovirus, Coxsackie virus, Dengue virus, Mumps virus, Poliovirus, Rabies virus, Rous sarcoma virus, Reovirus, Yellow fever virus, Zika virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, or Human Immunodeficiency virus type-2.

In some aspects, the methods disclosed herein are for treating infectious disease caused by a bacterial infection, wherein the bacterial infection comprises an infection of Mycobaterium tuberculosis, Mycobaterium bovis, Mycobaterium bovis strain BCG, BCG substrains, Mycobaterium avium, Mycobaterium intracellular, Mycobaterium africanum, Mycobaterium kansasii, Mycobaterium marinum, Mycobaterium ulcerans, Mycobaterium avium subspecies paratuberculosis, Nocardia asteroides, other Nocardia species, Legionella pneumophila, other Legionella species, Acetinobacter baumanii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii, other Bordetella species, Burkholderia mallei, Burkholderia psuedomallei, Burkholderia cepacian, Chlamydia pneumoniae, Chlamydia trachomatis, Chlamydia psittaci, Coxiella burnetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, Clostridium difficile, other Clostridium species, Yersinia enterolitica, and other Yersinia species, and Mycoplasma species.

In some aspects, the methods disclosed herein are for treating infectious disease caused by a fungal infection, wherein the fungal infection comprises an infection of Candida albicans, Cryptococcus neoformans, Histoplama capsulatum, Aspergillus fumigatus, Coccidiodes immitis, Paracoccidiodes brasiliensis, Blastomyces dermitidis, Pneumocystis carinii, Penicillium marneffi, or Alternaria alternate.

In some aspects, the methods disclosed herein are for treating infectious disease caused by a parasitic infection, wherein the parasitic infection comprises an infection of Toxoplasma gondii, Plasmodium falciparum, Plasmodium vivax, Plasmodium malariae, other Plasmodium species, Entamoeba histolytica, Naegleria fowleri, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Diphyllobothrium latum, Clonorchis sinensis; Clonorchis viverrini, Fasciola hepatica, Fasciola gigantica, Dicrocoelium dendriticum, Fasciolopsis buski, Metagonimus yokogawai, Opisthorchis viverrini, Opisthorchis felineus, Clonorchis sinensis, Trichomonas vaginalis, Acanthamoeba species, Schistosoma intercalatum, Schistosoma haematobium, Schistosoma japonicum, Schistosoma mansoni, other Schistosoma species, Trichobilharzia regenti, Trichinella spiralis, Trichinella britovi, Trichinella nelsoni, Trichinella nativa, or Entamoeba histolytica.

In some aspects, the γδ T cells administered in the method of any preceding aspect are formulated in a pharmaceutically acceptable carrier and a pharmaceutically acceptable excipient.

In some aspects, a method of any preceding aspect comprises administering the γδ T cells parenterally, intravenously, intraperitoneally, or subcutaneously, or through arterial infusion, venous infusion, or artificial catheter mediated infusion.

It should be further understood that any of the therapeutic methods described herein are also considered to be medical uses of any of the compositions disclosed herein, for treating any of the cancers or infectious diseases as disclosed herein.

### DESCRIPTION OF DRAWINGS

FIG. 1 shows effect of Fc on T cell expansion with CSTX-002 feeder cells. Inclusion of Fc on the CSTX-002 (K562-mb21-41BBL) cell line leads to an increased T cell expansion over day 14. Fc was anchored to the cellular membrane using neuraminidase (NA) stalk of different lengths NA2 being the shortest while NA4 the longest. The increase in T cell content was observed in all cultures stimulated with Fc-containing CSTX-002 cells and the longest NA fragment resulted in the highest final T cell content.

FIG. 2 shows that stimulation with CSTX-002-Fc leads to expansion of γδT cells. In a follow-up experiment using PBMCs derived from a different donor, it was confirmed that the inclusion of NA-Fc on CSTX-002 cells led to expansion of T cells. Phenotyping of the T cells demonstrated that large proportions of these cells consisted γδT cells with the Vδ2 cells preferentially expanding upon stimulation with NA-Fc expressing CSTX-002 feeder cells. The construct with the longest NA stock yielded the highest final content of Vδ2 cells.

FIG. 3 shows effect of the length of NA stalk and starting material on expansion of γδT cells. Longer NA stalk of the NA-Fc construct results in greater expansion of γδT cells of the Vδ2 subtype. NA4 is longer than NA2.

FIG. 4 shows Fc selectively induces expansion of γδT cells of the Vδ2 subtype. The expansion is not dependent on presence of NK cells. PBMCs obtained from four different donors were CD56-depleted (to remove NK cells) or not and stimulated with CSTX-002 cells that expressed or not Fc domain on the cell surface. Vδ2 T cell content was monitored periodically over the 14 day-culture time. FIG. 4 depicts a cumulative theoretical expansion of Vδ2 cells for all four donors. The inclusion of Fc on CSTX-002 cells led to expansion of T cells in all donors tested. Phenotyping of the T cells demonstrated that large proportions of these cells consisted γδT cells with the Vδ2 cells preferentially expanding upon stimulation with NA-Fc expressing CSTX-002 feeder cells. Depletion of NK cells did not negatively affect the expansion of Vδ2 cells.

FIG. 5 shows that Fc selectively induces expansion of γδT cells of the Vβ2 subtype. The expansion is not dependent on presence of NK cells. PBMCs obtained from two different donors were CD56-depleted (to remove NK cells) or not and stimulated with CSTX-002 cells that expressed or not Fc domain on the cell surface. Vδ2 T cell content was monitored periodically over the 14 day-culture time. The figure above depicts a cumulative theoretical expansion of Vδ2 cells on day 14. The inclusion of Fc on CSTX-002 cells led to significant expansion of Vδ2 T cells in all donors tested (p=001). Depletion of NK cells did not negatively affect the expansion of Vδ.

FIG. 6 shows that Fc selectively induces expansion of γδT cells of the Vδ2 subtype. The expansion is not dependent on presence of NK cells. PBMCs obtained from four different donors were CD56-depleted (to remove NK cells) or not and stimulated with CSTX-002 cells that expressed or not Fc domain on the cell surface. The figures depict example of the final cell content of a culture on day 14 for one of the donors. The inclusion of Fc on CSTX-002 cells led to significant increase in the content of Vδ2 T cells. Depletion of NK cells did not negatively affect the expansion of Vδ2 cells.

FIGS. 7A and 7B show the construction of a membrane-bound immune cell targeting ligand comprising an uncleaved signal anchor. FIG. 7A shows the structure of Type I and Type II integral membrane proteins that differ in the orientation with respect to their N- and C-termini. FIG. 7B shows the structure of the NA-Fc chimeric protein used as the membrane bound immune cell targeting ligand consisting of the neuraminidase transmembrane domain which serves as a membrane anchor, stalk region and human IgG′ Fc region.

FIG. 8 shows alternative constructions of membrane bound immune cell targeting ligands comprising an Fc domain comprising a neuraminidase (NA) signal anchor and increasing NA stalk lengths.

FIG. 9 shows an example of a membrane bound immune cell targeting ligand sequence, with an NA signal anchor fused to an IgG Fc domain by an RS linker.

FIGS. 10A-10B show amino acid acid sequence (FIG. 10A) alignment showing original, clone 1, and consensus sequence (SEQ ID NO: 36) and nucleic acid sequence (FIG. 10B) for NA1 -Fc.

FIGS. 11A-11B show amino acid acid sequence (FIG. 11A) and nucleic acid sequence (FIG. 11B) for NA2-Fc.

FIGS. 12A-12B show amino acid acid sequence (FIG. 12A) and nucleic acid sequence (FIG. 12B) for NA3-Fc.

FIGS. 13A-13B show amino acid acid sequence (FIG. 13A) and nucleic acid sequence (FIG. 13B) for NA4-Fc.

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### Definitions

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd Ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used herein, the terms “optional” or “optionally” mean that the subsequently described event or circumstance may or may not occur, and that the description includes instances where said event or circumstance occurs and instances where it does not.

The term “linker” refers at least a bivalent moiety with a site of attachment for a polypeptide and a site of attachment for another polypeptide. For example, a polypeptide can be attached to the linker at its N-terminus, its C-terminus or via a functional group on one of the side chains. The linker is sufficient to separate the two polypeptides by at least one atom and in some embodiments by more than one atom.

As used herein, “N-terminal side” or “amino terminal end” refers to directionality of a peptide, polypeptide, or protein and may not mean the N-terminus. In some aspects, where a chimeric or fusion peptide, polypeptide, or protein is discussed, the N-terminal side may refer only to a component of the chimeric or fusion peptide, polypeptide, or protein and not the entire structure. For example, where a Fc domain is discussed, and the Fc domain is described as fused with its amino terminal end or N-terminal side facing intracellularly, contemplated herein are chimeric or fusion peptides, polypeptides, or proteins wherein the signal anchor is at the N-terminus of the chimeric or fusion construct and actually spans the cellular membrane. Thus, in such a chimera, the trans-membrane anchor is attached to the amino terminal side of the Fc domain, with the directionality of the Fc domain has the N-terminal side facing the cell which is inverted relative to an Fc domain on a typical B cell which would typically have the carboxy end spanning the cellular membrane and amino terminal end extending to the extracellular matrix.

The terms “peptide,” “polypeptide” and “protein” are used interchangeably to refer to a polymer of amino acid residues.

The term “sequence identity” as used herein, indicates a quantitative measure of the degree of identity between two sequences of substantially equal length. The percent identity of two sequences, whether nucleic acid or amino acid sequences, is the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found on the GenBank website. In general, the substitutions are conservative amino acid substitutions: limited to exchanges within members of group 1: glycine, alanine, valine, leucine, and Isoleucine; group 2: serine, cysteine, threonine, and methionine; group 3: proline; group 4: phenylalanine, tyrosine, and tryptophan; group 5: aspartate, glutamate, asparagine, and glutamine

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity.

As various changes could be made in the above-described cells and methods without departing from the scope of the invention, it is intended that all matter contained in the above description and in the examples given below, shall be interpreted as illustrative and not in a limiting sense.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration, but are undetectable or detectable at negligible amounts in distal parts of the subject's body. Administration includes self-administration and the administration by another. In some embodiments, the compositions disclosed herein are administered parenterally, intravenously, intraperitoneally, or subcutaneously, or through arterial infusion, venous infusion, or artificial catheter mediated infusion.

“Treat,” “treating,” “treatment,” and grammatical variations thereof as used herein, include the administration of a composition with the intent or purpose of partially or completely preventing, delaying, curing, healing, alleviating, relieving, altering, remedying, ameliorating, improving, stabilizing, mitigating, and/or reducing the intensity or frequency of one or more a diseases or conditions, a symptom of a disease or condition, or an underlying cause of a disease or condition. Treatments according to the invention may be applied preventively, prophylactically, pallatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for day(s) to years prior to the manifestation of symptoms of a disease or an infection.

### Compositions and Methods

The invention here conceives of are compositions and uses thereof of expanding γδ T cells that includes Fc domain of an antibody, that is competent for agonizing a Fc receptor (e.g., CD16), bound to a feeder cells, engineered particles, exosomes, or on some other solid support. The feeder cells, engineered particles, exosomes and other solid supports with bound Fc domain can also be comprised with other stimulating factors such as membrane bound IL-21, 4-1BBL, other cytokines, or other chemical moieties that simultaneously engage other stimulatory (or possibly inhibitory) receptors and corresponding signaling pathways. As noted above, challenges remain to utilize γδ T cells in clinical application. The compositions and the methods disclosed herein show surprisingly effect in inducing, activating, and/or expanding γδ T cells in vivo and/or in vitro. The expanded γδ T cells are effective for treating diseases, such as cancers or infectious diseases.

### I. Peptides

Accordingly, disclosed herein are γδ T cell expanding compositions, wherein the compositions are engineered feeder cells, engineered plasma membrane particles, exosomes, and engineered lymphocytes (such as, for example lymphocytes (such as T cells) engineered to express Fc domains to stimulate γδ T cells) and solid supports comprising a membrane bound Fc fusion peptide (referred to herein as Fc-bound feeder cells, Fc-bound engineered plasma membrane particles, and Fc-bound exosomes, Fc-bound lymphocytes, respectively) wherein the Fc fusion peptide comprises a transmembrane peptide domain linked to the amino terminus or the carboxyl terminus of an Fc domain. In one aspect, the transmembrane domain of the Fc fusion peptide can comprise a cleaved or uncleaved signal anchor sequence such as the transmembrane domain of neuraminidase, the signal-anchor from parainfluenza virus hemagglutinin-neuraminidase, the signal-anchor from the transferrin receptor, the signal-anchor from the MHC class II invariant chain, the signal-anchor from P glycoprotein, the signal-anchor from asialoglycoprotein receptor, or the signal-anchor from a neutral endopeptidase. In one example, the transmembrane domain comprises a parainfluenza virus hemagglutinin-neuraminidase (NA) peptide sequence. The transmembrane neuraminidase (NA) peptide domain may couple or bind the Fc domain to the external surface of a feeder cell. In other aspects, the transmembrane neuraminidase (NA) peptide domain is used to couple or bind the Fc domain to the external surface of an engineered feeder cell, an engineered plasma membrane nanoparticle, exosome or a solid support. In some embodiments, the NA peptide domain consists of the N-terminal cytoplasmic tail, an uncleaved signal-anchor which serves as a transmembrane domain, and a stalk region which extends from the plasma membrane. It will be understood that the length of the stalk region can be varied, wherein the length of the stalk region affects the efficacy of the surface-bound Fc domain-NA peptide in stimulating γδ T cell expansion.

In some embodiments, the transmembrane domain comprises a parainfluenza virus hemagglutinin-neuraminidase (NA) peptide sequence. In some embodiments, the NA peptide domain comprises a sequence at least 80%, 81%, 82%, 83%, 84%, 85%, 86%, 87%, 88%, 89%, 90%, 91%, 92%, 93%, 94%, 95%, 96%, 97%, 98, or 99% sequence identity with SEQ ID NO: 1, SEQ ID NO: 2, SEQ ID NO: 3, or SEQ ID NO: 4. The transmembrane domain and the Fc domain may be linked via a peptide linker.

The Fc domain is the ligand to which the γδ T cell surface receptor CD16 (Fc γRIII) binds. CD16 is one of the primary receptors on γδ T cells that binds to the Fc portion of an antibody (for example, an IgG1, IgG2, IgG3, and/or IgG4 Fc domain. In another aspect, Fc domain (IgG1, IgG2, IgG3, and/or IgG4 can also bind the CD16 receptor on other immune cells, such as mast cells, macrophages, or antigen presenting cells. In another aspect, other types of cells can be engineered to be Fc-bound. The present disclosure Fc-bound engineered feeder cells, Fc-bound engineered plasma membrane particles, Fc-bound exosomes, or Fc-bound solid supports.

In some embodiments, the Fc domain comprises an immunoglobulin Fc domain selected from IgG1, IgG2, IgG3, IgG4, IgA and IgE. In some embodiment, the Fc domain binds to CD16.

In another aspect, other Fc immunoglobulin isotypes (IgA, IgE, IgM) other than IgG, could be used to stimulate the respectively corresponding different Fc receptors for stimulation of other immune cell types. For example, the domain FcαRI (CD89) specifically binds to IgA on macrophages, neutrophils, eosinophils; FcγRI (CD64) specifically binds to IgG on monocytes and macrophages; and FcεRII (CD23) specifically binds to IgE on B cells. Fc binds to CD64 on monocytes or macrophages and thus stimulates them. Thus, the fusion peptides, Fc-bound feeder cells (FCs), Fc bound lymphocytes, Fc-bound engineered plasma membrane (PM) particles, Fc-bound engineered exosomes and compositions containing them can also be used to expand mast cells and/or macrophages substantially according to the methods described herein for expanding γδ T cells.

In one aspect, disclosed herein are fusion peptides comprising an immunoglobulin Fc domain (for example, an IgG1, IgG2, IgG3, IgG4, IgA and/or IgE Fc domain) fused to a transmembrane domain, for example an NA peptide domain, as described above. The Fc domain(s) can be presented as a monomeric, dimeric, or multimeric construct. In one aspect, the Fc domain(s) can be further modified to optimize or enhance expansion and/or activation of γδ T cells. For example, the Fc domain(s) can be modified to increase affinity for CD16. Thus, for example, the Fc domain(s) may comprise one or more mutations such as, for example, T256A, K290A, S298A, E333A, K334A, L235V, F243L, R292P, Y300L, and/or P396L. Similarly, the Fc domain(s) can be further modified to increase selectivity of binding to the activating (IIIa) vs, inhibitory Fc(IIb) receptor. Thus, for example, the Fc domain(s) may comprise one, two, three, four, five, six, seven, eight or more mutations or alternative forms such as, for example, S239D, I332E, A330L, F243L, R292P, V305I, and/or P396L. For example, in one aspect, the Fc domain can be modified to comprise R292L, Y300L, V305I, and P396L. In another example, the Fc domain can be modified to comprise S239D, I332E, and A330L.

The transmembrane domain, for example an NA peptide domain can be linked directly to the Fc domain via a chemical bond, or indirectly via a linker. A direct chemical bond is for example a covalent bond (e.g., peptide bond, ester bond, or the like), or alternatively, a non-covalent bond (e.g., ionic, electrostatic, hydrogen, hydrophobic, Van der interactions, or π-effects). An indirect link can be achieved using a linker, i.e., a chemical group that connects one or more other chemical groups via at least one covalent bond. Suitable linkers include amino acids, peptides, nucleotides, nucleic acids, dimeric hinged Fc, organic linker molecules (e.g., maleimide derivatives, N-ethoxybenzylimidazole, biphenyl-3,4′,5-tricarboxylic acid, p-aminobenzyloxycarbonyl, and the like), disulfide linkers, and polymer linkers (e.g., PEG). The linker can include one or more spacing groups including, but not limited to alkylene, alkenylene, alkynylene, alkyl, alkenyl, alkynyl, alkoxy, aryl, heteroaryl, aralkyl, aralkenyl, aralkynyl and the like. The linker can be neutral, or carry a positive or negative charge. Additionally, the linker can be cleavable such that the linker's covalent bond that connects the linker to another chemical group can be broken or cleaved under certain conditions, including pH, temperature, salt concentration, light, a catalyst, or an enzyme. In one aspect, the NA peptide domain can be an NA4-fc Siadel (S239D/I332E/A330L).

In one aspect, the linker may be a peptide linker. Examples of suitable peptide linkers are well known in the art, and programs to design linkers are readily available (see, e.g., Crasto et al., Protein Eng., 2000, 13(5):309-312). The peptide linker can, for example, be a restriction site linker such as the short sequence RS, or a flexible amino acid linker (e.g., comprising small, non-polar or polar amino acids). Non-limiting examples of flexible linkers include LEGGGS (SEQ ID NO: 5), TGSG (SEQ ID NO:6), GGSGGGSG (SEQ ID NO:7), (GGGGS)₁₋₄ SEQ ID NO: 8), (GGGS)₁₋₄ (SEQ ID NO: 9), (GSGGGG)₁₋₄ (SEQ ID NO: 10), and (Gly)₆₋₈ (SEQ ID NO: 11). Alternatively, the peptide linker can be a rigid amino acid linker. Such linkers include (EAAAK)₁₋₄ (SEQ ID NO: 12), A(EAAAK)₂₋₅A (SEQ ID NO: 13), PAPAP (SEQ ID NO: 14), and (AP)₆₋₈ (SEQ ID NO:15). The Fc domain can be linked to the N-terminus, the C-terminus, and/or to an internal location of the NA peptide. In one aspect, a peptide linker may be a short amino acid sequence of 3, 4, 5, 6, 7, 8, 9, 10, 11, 12, 13, 14, 15, 16, 17, 18, 19 or 20 amino acids. In another aspect, a peptide linker may be an amino acid sequence of any of 2-10, 2-8 or 2-6 amino acids in length.

In some embodiments, the Fc fusion peptide has an amino acid sequence having at least about 80%, 81%, 82%, 83%, 84%, 85%, 86%, 87%, 88%, 89%, 90%, 91%, 92%, 93%, 94%, 95%, 96%, 97%, 98, or 99%

(SEQ ID NO: 16)
MNPNQKITTIGSICLVVGLISLILQIGNIISIWISHSIQT
GSQNHTGICNRSDKTHTCPPCPAPELLGGPSVFLFPPKPK
DTLMISRTPEVTCVVVDVSHEDPEVKFNWYVDGVEVHNAK
TKPREEQYNSTYRVVSVLTVLHQDWLNGKEYKCKVSNKAL
PAPIEKTISKAKGQPREPQVYTLPPSREEMTKNQVSLTCL
VKGFYPSDIAVEWESNGQPENNYKTTPPVLDSDGSFFLYS
KLTVDKSRWQQGNVFSCSVMHEALHNHYTQKSLSLSPGK.

In some embodiments, the Fc fusion peptide has an amino acid sequence having at least about 80%, 81%, 82%, 83%, 84%, 85%, 86%, 87%, 88%, 89%, 90%, 91%, 92%, 93%, 94%, 95%, 96%, 97%, 98, or 99% sequence identity with

(SEQ ID NO: 17)
MNPNQKITTIGSICLVVGLISLILQIGNIISIWISHSIQT
GSQNHTGICNQNIITYKNSTWVKDTTSVILTGNSSLCPIR
RSDKTHTCPPCPAPELLGGPSVFLFPPKPKDTLMISRTPE
VTCVVVDVSHEDPEVKFNWYVDGVEVHNAKTKPREEQYNS
TYRVVSVLTVLHQDWLNGKEYKCKVSNKALPAPIEKTISK
AKGQPREPQVYTLPPSREEMTKNQVSLTCLVKGFYPSDIA
VEWESNGQPENNYKTTPPVLDSDGSFFLYSKLTVDKSRWQ
QGNVFSCSVMHEALHNHYTQKSLSLSPGK.

In some embodiments, the Fc fusion peptide has an amino acid sequence having at least about 80%, 81%, 82%, 83%, 84%, 85%, 86%, 87%, 88%, 89%, 90%, 91%, 92%, 93%, 94%, 95%, 96%, 97%, 98, or 99% sequence identity with

(SEQ ID NO: 18)
MNPNQKITTIGSICLVVGLISLILQIGNIISIWISHSIQT
GSQNHTGICNQNIITYKNSTWVKDTTSVILTGNSSLCPIR
GWAIYSKDNSIRIGSKGDVFRSDKTHTCPPCPAPELLGGP
SVFLFPPKPKDTLMISRTPEVTCVVVDVSHEDPEVKFNWY
VDGVEVHNAKTKPREEQYNSTYRVVSVLTVLHQDWLNGKE
YKCKVSNKALPAPIEKTISKAKGQPREPQVYTLPPSREEM
TKNQVSLTCLVKGFYPSDIAVEWESNGQPENNYKTTPPVL
DSDGSFFLYSKLTVDKSRWQQGNVFSCSVMHEALHNHYTQ
KSLSLSPGK.

In some embodiments, the Fc fusion peptide has an amino acid sequence having at least about 80%, 81%, 82%, 83%, 84%, 85%, 86%, 87%, 88%, 89%, 90%, 91%, 92%, 93%, 94%, 95%, 96%, 97%, 98, or 99% sequence identity with

(SEQ ID NO: 19)
MNPNQKITTIGSICLVVGLISLILQIGNIISIWISHSIQT
GSQNHTGICNQNIITYKNSTWVKDTTSVILTGNSSLCPIR
GWAIYSKDNSIRIGSKGDVFVIREPFISCSHLECRTFFLT
RSDKTHTCPPCPAPELLGGPSVFLFPPKPKDTLMISRTPE
VTCVVVDVSHEDPEVKFNWYVDGVEVHNAKTKPREEQYNS
TYRVVSVLTVLHQDWLNGKEYKCKVSNKALPAPIEKTISK
AKGQPREPQVYTLPPSREEMTKNQVSLTCLVKGFYPSDIA
VEWESNGQPENNYKTTPPVLDSDGSFFLYSKLTVDKSRW
QQGNVFSCSVMHEALHNHYTQKSLSLSPGK.

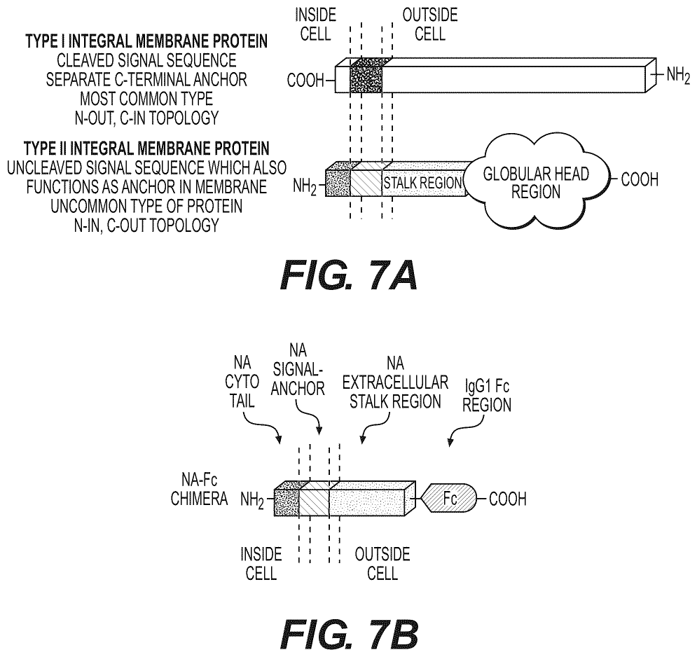

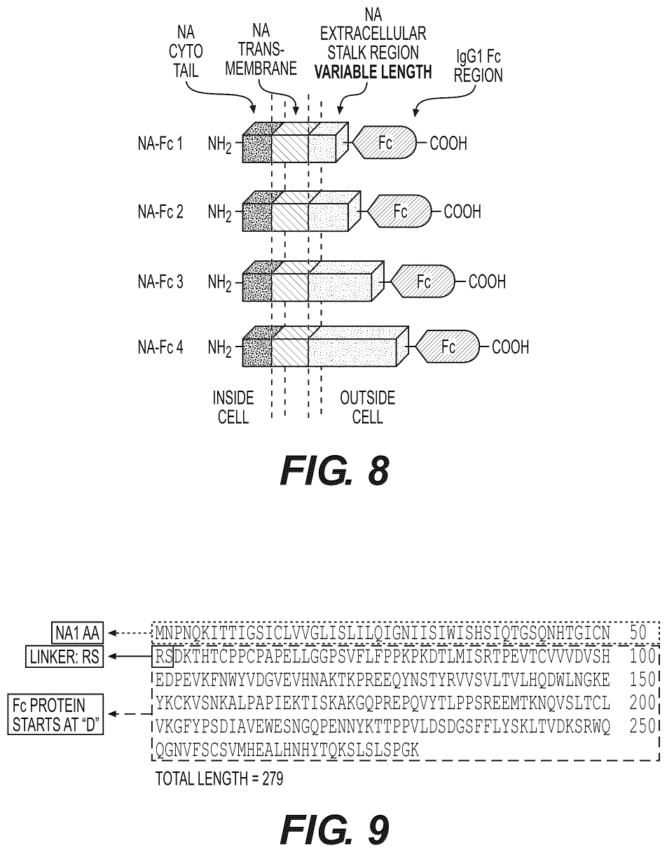

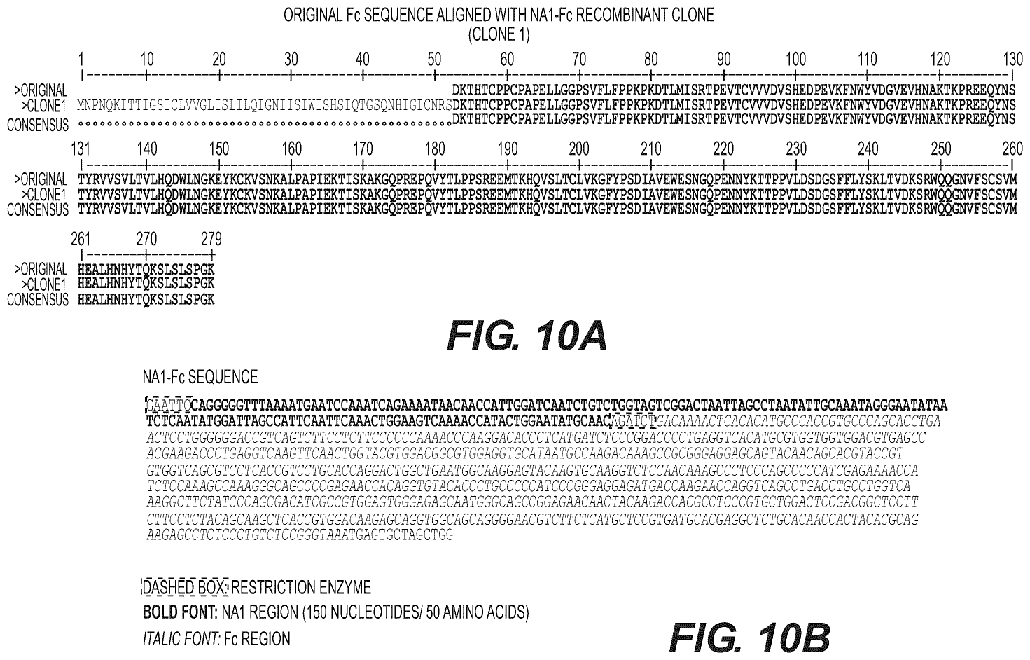

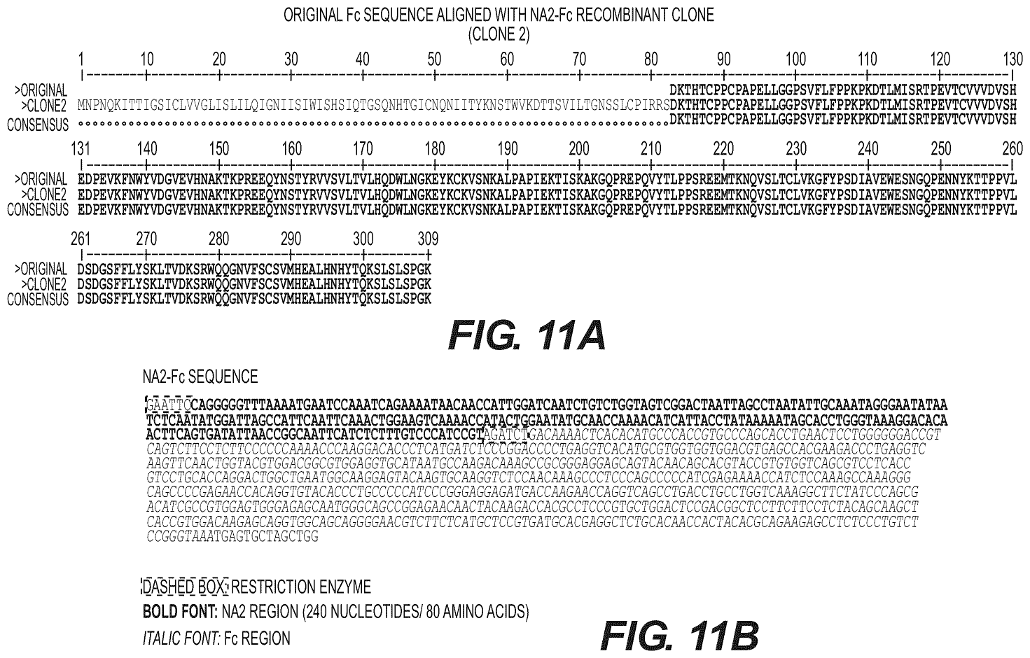

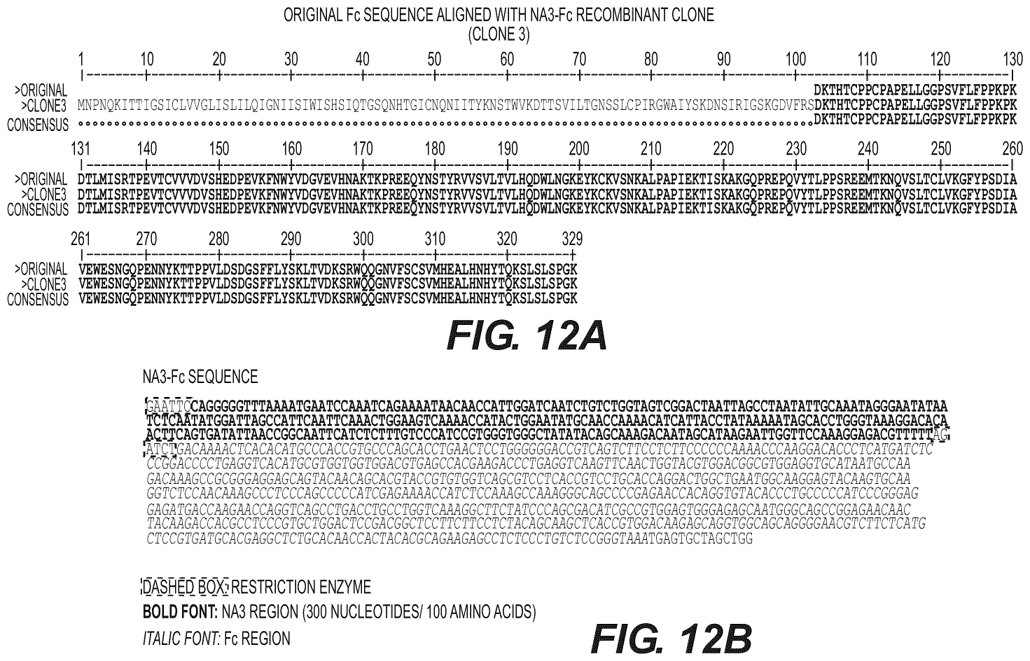

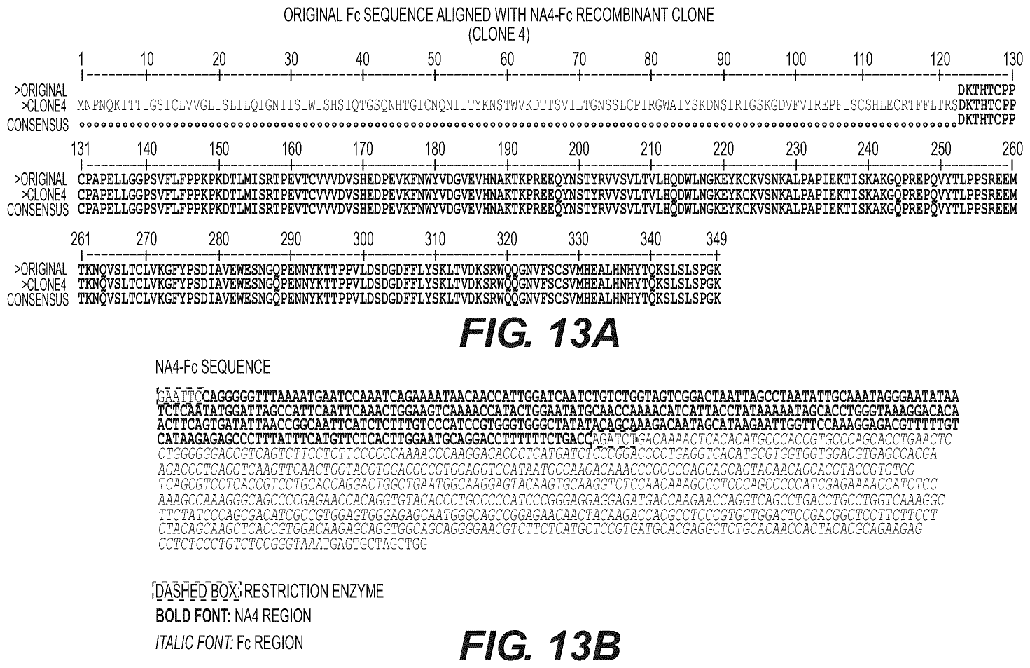

To target placement of the Fc domain on the plasma membrane, membrane targeting domain from the well characterized influenza virus neuraminidase protein (NA) can be used which consists of the N-terminal cytoplasmic tail, an uncleaved signal-anchor which serves as a transmembrane domain, and a stalk region which extends from the plasma membrane. FIGS. 7A and 7B are schematics showing the construction of a membrane bound immune cell targeting ligand comprising an uncleaved signal anchor sequence. FIG. 7A shows the structure of Type I and Type II integral membrane proteins and the signal anchors for each. FIG. 7B shows the structure of the uncleaved signal anchor from a Type II integral membrane protein used in the membrane bound immune cell targeting ligand. As shown in FIG. 7B, an exemplary but non-limiting construct according to the present disclosure is comprised of an NA-Fc chimera where the Fc domain (IgG1) is linked via a short linker to the uncleaved NA stalk region. Notably, the NA-Fc chimera can be inserted into recombinant P/V/F virus to generate a novel oncolytic virus which is specific for tumor versus normal cells (due to P/V mutations) and can enhance ADCC by NK cells. FIG. 8 shows alternative constructions of an NA-Fc chimera with increasing NA stalk lengths.

The NA-Fc construct can comprise the NA peptide domain (SEQ ID NO: 1), a linker (for example an RS linker), a hinge region DKTHTCPPCPAPELL (SEQ ID NO: 20) or TCPPCPAPELL (SEQ ID NO: 21), and an Fc region

(SEQ ID NO: 22)
GGPSVFLFPPKPKDTLMISRTPEVTCVVVDVSHEDPEVKF
NWYVDGVEVHNAKTKPREEQYNSTYRVVSVLTVLHQDWLN
GKEYKCKVSNKALPAPIEKTISKAKGQPREPQVYTLPPSR
EEMTKNQVSLTCLVKGFYPSDIAVEWESNGQPENNYKTT
PPVLDSDGSFFLYSKLTVDKSRWQQGNVFSCSVMHEALHN
HYTQKSLSLSPGK

comprising a CH2 domain

(SEQ ID NO: 23)
GGPSVFLFPPKPKDTLMISRTPEVTCVVVDVSHEDPEVKF
NWYVDGVEVHNAKTKPREEQYNSTYRVVSVLTVLHQDWLN
GKEYKCKVSNKALPAPIEKTISKAK

and a CH3 domain

(SEQ ID NO: 24)
GQPREPQVYTLPPSREEMTKNQVSLTCLVKGFYPSDIAVE
WESNGQPENNYKTTPPVLDSDGSFFLYSKLTVDKSRWQQG
NVFSCSVMHEALHNHYTQKSLSLSPGK.

It is understood and herein contemplated that an NA-Fc chimera can include any length of the membrane targeting domain from the well characterized influenza virus neuraminidase protein (NA) including

(SEQ ID NO: 2)
MNPNQKITTIGSICLVVGLISLILQIGNIISIWISHSIQT
GSQNHTGICNQNIITYKNSTWVKDTTSVILTGNSSLCPIR,
(SEQ ID NO: 3)
MNPNQKITTIGSICLVVGLISLILQIGNIISIWISHSIQT
GSQNHTGICNQNIITYKNSTWVKDTTSVILTGNSSLCPIR
GWAIYSKDNSIRIGSKGDVF,
(SEQ ID NO: 4)
MNPNQKITTIGSICLVVGLISLILQIGNIISIWISHSIQT
GSQNHTGICNQNIITYKNSTWVKDTTSVILTGNSSLCPIR
GWAIYSKDNSIRIGSKGDVFVIREPFISCSHLECRTFFLT.

As noted above, the Fc region can comprise one or more mutations such as, for example, L234Y, L235V, L235Q, G236W, S239D, S239M, F243L, T256A, K290A, R292P, N297Q, S298A, Y300L, V305I, A330L, I332E, E333A, K334A, and/or P396L. Thus, specifically disclosed herein are Fc regions comprising a Leucine (L) or Tyrosine (Y) at residue 234, a Leucine (L), Glutamine, or Valine (V) at residue 235, a Glutamine (G) or Tryptophan (W) at residue 236, a Serine (S), Methionine (M), or Aspartate (D) at residue 239, and Phenylalanine (F) or Leucine (L) at residue 243, a threonine (T) or Alanine (A) at residue 256, a Histidine (H) or Aspartate (D) at residue 268, an Aspartate (D) or Glutamate (E) at residue 270, a Lysine (K) or Alanine (A) at residue 290, an Arginine (R) or Proline (P) at residue 292, a Serine (S) or Alanine (A) at residue 298, an Asparagine or Glutamine at residue 297, a Tyrosine (Y) or Leucine (L) at residue 300, a Valine (V) or Isoleucine (I) at residue 305, a Lysine (K) or Aspartate (D) at residue 326, an Alanine (A), Methionine (M), or Leucine (L) at residue 330, and Isoleucine (I) or Glutamate (E) at residue 332, a Glutamate (E) or Alanine (A) at residue 333, a Lysine (K), Glutamate (E), or Alanine (A) at residue 334, and/or a Proline (P) or Leucine (L) at residue 396. It is specifically understood that no substitution or any one or combination two, three, four, five, six, seven, eight, nine, ten, eleven, twelve, thirteen, fourteen, fifteen, sixteen, or seventeen of the substitutions mentioned herein can be present in the Fc region. Accordingly, in one aspect disclosed herein are fusion proteins comprising a substitution of the Fc region at F243L, R292P, Y300L, V305I, and P396L where the sequence of the NA4-Fc comprises

(SEQ ID NO: 25)
MNPNQKITTIGSICLVVGLISLILQIGNIISIWISHSIQT
GSQNHTGICNQNIITYKNSTWVKDTTSVILTGNSSLCPIR
GWAIYSKDNSIRIGSKGDVFVIREPFISCSHLECRTFFLT
DKTHTCPPCPAPELLGGPSVFLFPPKPKDTLMISRTPEVT
CVVVDVSHEDPEVKFNWYVDGVEVHNAKTKPREEQYNSTY
RVVSVLTVLHQDWLNGKEYKCKVSNKALPAPIEKTISKAK
GQPREPQVYTLPPSREEMTKNQVSLTCLVKGFYPSDIAV
EWESNGQPENNYKTTPPVLDSDGSFFLYSKLTVDKSRWQQ
GNVFSCSVMHEALHNHYTQKSLSLSPGK.

In one aspect, the NA4-Fc fusion comprises S293D, I332E, and A330L substitutions having an Fc domain with the sequence

(SEQ ID NO: 26)
GGPDVFLFPPKPKDTLMISRTPEVTCVVVDVSHEDPEVKF
NWYVDGVEVHNAKTKPREEQYNSTYRVVSVLTVLHQDWLN
GKEYKCKVSNKALPLPEEKTISKAKGQPREPQVYTLPPSR
EEMTKNQVSLTCLVKGFYPSDIAVEWESNGQPENNYKTTP
PVLDSDGSFFLYSKLTVDKSRWQQGNVFSCSVMHEALHNH
YTQKSLSLSPG

which comprises a CH2 domain with the sequence

(SEQ ID NO: 27)
GGPDVFLFPPKPKDTLMISRTPEVTCVVVDVSHEDPEVKF
NWYVDGVEVHNAKTKPREEQYNSTYRVVSVLTVLHQDWLN
GKEYKCKVSNKALPLPEEKTISKAK

and a complete sequence of

(SEQ ID NO: 28)
MNPNQKITTIGSICLVVGLISLILQIGNIISIWISHSIQT
GSQNHTGICNQNIITYKNSTWVKDTTSVILTGNSSLCPIR
GWAIYSKDNSIRIGSKGDVFVIREPFISCSHLECRTFFLT
DKTHTCPPCPAPELLGGPDVFLFPPKPKDTLMISRTPEVT
CVVVDVSHEDPEVKFNWYVDGVEVHNAKTKPREEQYNSTY
RVVSVLTVLHQDWLNGKEYKCKVSNKALPLPEEKTISKAK
GQPREPQVYTLPPSREEMTKNQVSLTCLVKGFYPSDIAV
EWESNGQPENNYKTTPPVLDSDGSFFLYSKLTVDKSRWQQ
GNVFSCSVMHEALHNHYTQKSLSLSPGK.

In another example, the Fc region of the NA-Fc fusion comprises a CH2 domain with F243, R292, Y300, and V305 as set for in SEQ ID NO: 23

GGPSVFLFPPKPKDTLMISRTPEVTCVVVDVSHEDPEVKFNWYVDGVEVHNAKTKPRE EQYNSTYRVVSVLTVLHQDWLNGKEYKCKVSNKALPAPIEKTISKAK and a P396 in the CH3 domain as set forth in SEQ ID NO: 24
GQPREPQVYTLPPSREEMTKNQVSLTCLVKGFYPSDIAVEWESNGQPENNYKTTPPVLD SDGSFFLYSKLTVDKSRWQQGNVFSCSVMHEALHNHYTQKSLSLSPGK thereby having an Fc region with the sequence

(SEQ ID NO: 22)
GGPSVFLFPPKPKDTLMISRTPEVTCVVVDVSHEDPEVKF
NWYVDGVEVHNAKTKPREEQYNSTYRVVSVLTVLHQDWLN
GKEYKCKVSNKALPAPIEKTISKAKGQPREPQVYTLPPSR
EEMTKNQVSLTCLVKGFYPSDIAVEWESNGQPENNYKTT
PPVLDSDGSFFLYSKLTVDKSRWQQGNVFSCSVMHEALHN
HYTQKSLSLSPGK.

In one aspect, the NA-Fc fusion can comprise the sequence

(SEQ ID NO: 29)
MNPNQKITTIGSICLVVGLISLILQIGNIISIWISHSIQT
GSQNHTGICNQNIITYKNSTWVKDTTSVILTGNSSLCPIR
GWAIYSKDNSIRIGSKGDVFVIREPFISCSHLECRTFFLT
DKTHTCPPCPAPELLGGPSVFLFPPKPKDTLMISRTPEVT
CVVVDVSHEDPEVKFNWYVDGVEVHNAKTKPREEQYNSTY
RVVSVLTVLHQDWLNGKEYKCKVSNKALPAPIEKTISKAK
GQPREPQVYTLPPSREEMTKNQVSLTCLVKGFYPSDIAV
EWESNGQPENNYKTTPPVLDSDGSFFLYSKLTVDKSRWQQ
GNVFSCSVMHEALHNHYTQKSLSLSPGK.

In another example, the Fc region of the NA-Fc fusion comprises a CH2 domain with F243L, R292P, Y300L, and V305I substitutions as set for in SEQ ID NO: 23
GGPSVFLFPPKPKDTLMISRTPEVTCVVVDVSHEDPEVKFNWYVDGVEVHNAKTKPRE EQYNSTYRVVSVLTVLHQDWLNGKEYKCKVSNKALPAPIEKTISKAK and a P396L substation in the CH3 domain as set forth in SEQ ID NO: 24
GQPREPQVYTLPPSREEMTKNQVSLTCLVKGFYPSDIAVEWESNGQPENNYKTTPPVLD SDGSFFLYSKLTVDKSRWQQGNVFSCSVMHEALHNHYTQKSLSLSPGK and a full-length Fc region with the sequence

(SEQ ID NO: 22)
GGPSVFLFPPKPKDTLMISRTPEVTCVVVDVSHEDPEVKF
NWYVDGVEVHNAKTKPREEQYNSTYRVVSVLTVLHQDWLN
GKEYKCKVSNKALPAPIEKTISKAKGQPREPQVYTLPPSR
EEMTKNQVSLTCLVKGFYPSDIAVEWESNGQPENNYKTT
PPVLDSDGSFFLYSKLTVDKSRWQQGNVFSCSVMHEALHN
HYTQKSLSLSPGK

and an NA-Fc fusion with the sequence

(SEQ ID NO: 30)
MNPNQKITTIGSICLVVGLISLILQIGNIISIWISHSIQT
GSQNHTGICNQNIITYKNSTWVKDTTSVILTGNSSLCPIR
GWAIYSKDNSIRIGSKGDVFVIREPFISCSHLECRTFFLT
DKTHTCPPCPAPELLGGPSVFLFPPKPKDTLMISRTPEVT
CVVVDVSHEDPEVKFNWYVDGVEVHNAKTKPREEQYNSTY
RVVSVLTVLHQDWLNGKEYKCKVSNKALPAPIEKTISKAK
GQPREPQVYTLPPSREEMTKNQVSLTCLVKGFYPSDIAV
EWESNGQPENNYKTTPPVLDSDGSFFLYSKLTVDKSRWQQ
GNVFSCSVMHEALHNHYTQKSLSLSPGK.

In one aspect, the NA4-Fc fusion protein can comprise 2 Fc domains linked via a hinge region. For example, the NA-Fc fusion can comprise the sequence

(SEQ ID NO: 31)
MNPNQKITTIGSICLVVGLISLILQIGNIISIWISHSIQT
GSQNHTGICNQNIITYKNSTWVKDTTSVILTGNSSLCPIR
GWAIYSKDNSIRIGSKGDVFVIREPFISCSHLECRTFFLT
DKTHTCPPCPAPELLGGPSVFLFPPKPKDTLMISRTPEVT
CVVVDVSHEDPEVKFNWYVDGVEVHNAKTKPREEQYNSTY
RVVSVLTVLHQDWLNGKEYKCKVSNKALPAPIEKTISKAK
GQPREPQVYTLPPSREEMTKNQVSLTCLVKGFYPSDIAV
EWESNGQPENNYKTTPPVLDSDGSFFLYSKLTVDKSRWQQ
GNVFSCSVMHEALHNHYTQKSLSLSPGKGGPSVFLFPPKP
KDTLMISRTPEVTCVVVDVSHEDPEVKFNWYVDGVEVHNA
KTKPREEQYNSTYRVVSVLTVLHQDWLNGKEYKCKVSNKA
LPAPIEKTISKAKGQPREPQVYTLPPSREEMTKNQVSLTC
LVKGFYPSDIAVEWESNGQPENNYKTTPPVLDSDGSFFLY
SKLTVDKSRWQQGNVFSCSVMHEALHNHYTQKSLSLSPGK.

In another aspect, the Fc domains can be asymmetric variants, for example, one heavy chain Fc domain can comprise L234Y/L235Q/G236W/S239M/H268D/D270E/S298A while the other Fc domain comprises D270E/K326D/A330M/K334E.

In general, any amino acid substitution is conservative, i.e., limited to exchanges within members of group 1: glycine, alanine, valine, leucine, and Isoleucine; group 2: serine, cysteine, threonine, and methionine; group 3: proline; group 4: phenylalanine, tyrosine, and tryptophan; and group 5: aspartate, glutamate, asparagine, and glutamine

In some embodiments, the NA-Fc fusion is encoded by

(SEQ ID NO: 32)
GAATTCCAGGGGGTTTAAAATGAATCCAAATCAGAAAATA
ACAACCATTGGATCAATCTGTCTGGTAGTCGGACTAATTA
GCCTAATATTGCAAATAGGGAATATAATCTCAATATGGAT
TAGCCATTCAATTCAAACTGGAAGTCAAAACCATACTGGA
ATATGCAACAGATCTGACAAAACTCACACATGCCCACCGT
GCCCAGCACCTGAACTCCTGGGGGGACCGTCAGTCTTCCT
CTTCCCCCCAAAACCCAAGGACACCCTCATGATCTCCCGG
ACCCCTGAGGTCACATGCGTGGTGGTGGACGTGAGCCACG
AAGACCCTGAGGTCAAGTTCAACTGGTACGTGGACGGCGT
GGAGGTGCATAATGCCAAGACAAAGCCGCGGGAGGAGCAG
TACAACAGCACGTACCGTGTGGTCAGCGTCCTCACCGTCC
TGCACCAGGACTGGCTGAATGGCAAGGAGTACAAGTGCAA
GGTCTCCAACAAAGCCCTCCCAGCCCCCATCGAGAAAACC
ATCTCCAAAGCCAAAGGGCAGCCCCGAGAACCACAGGTGT
ACACCCTGCCCCCATCCCGGGAGGAGATGACCAAGAACCA
GGTCAGCCTGACCTGCCTGGTCAAAGGCTTCTATCCCAGC
GACATCGCCGTGGAGTGGGAGAGCAATGGGCAGCCGGAGA
ACAACTACAAGACCACGCCTCCCGTGCTGGACTCCGACGG
CTCCTTCTTCCTCTACAGCAAGCTCACCGTGGACAAGAGC
AGGTGGCAGCAGGGGAACGTCTTCTCATGCTCCGTGATGC
ACGAGGCTCTGCACAACCACTACACGCAGAAGAGCCTCTC
CCTGTCTCCGGGTAAATGAGTGCTAGCTGG,
(SEQ ID NO: 33)
GAATTCCAGGGGGTTTAAAATGAATCCAAATCAGAAAATA
ACAACCATTGGATCAATCTGTCTGGTAGTCGGACTAATTA
GCCTAATATTGCAAATAGGGAATATAATCTCAATATGGAT
TAGCCATTCAATTCAAACTGGAAGTCAAAACCATACTGGA
ATATGCAACCAAAACATCATTACCTATAAAAATAGCACCT
GGGTAAAGGACACAACTTCAGTGATATTAACCGGCAATTC
ATCTCTTTGTCCCATCCGTAGATCTGACAAAACTCACACA
TGCCCACCGTGCCCAGCACCTGAACTCCTGGGGGGACCGT
CAGTCTTCCTCTTCCCCCCAAAACCCAAGGACACCCTCAT
GATCTCCCGGACCCCTGAGGTCACATGCGTGGTGGTGGAC
GTGAGCCACGAAGACCCTGAGGTCAAGTTCAACTGGTACG
TGGACGGCGTGGAGGTGCATAATGCCAAGACAAAGCCGCG
GGAGGAGCAGTACAACAGCACGTACCGTGTGGTCAGCGTC
CTCACCGTCCTGCACCAGGACTGGCTGAATGGCAAGGAGT
ACAAGTGCAAGGTCTCCAACAAAGCCCTCCCAGCCCCCAT
CGAGAAAACCATCTCCAAAGCCAAAGGGCAGCCCCGAGAA
CCACAGGTGTACACCCTGCCCCCATCCCGGGAGGAGATGA
CCAAGAACCAGGTCAGCCTGACCTGCCTGGTCAAAGGCTT
CTATCCCAGCGACATCGCCGTGGAGTGGGAGAGCAATGGG
CAGCCGGAGAACAACTACAAGACCACGCCTCCCGTGCTGG
ACTCCGACGGCTCCTTCTTCCTCTACAGCAAGCTCACCGT
GGACAAGAGCAGGTGGCAGCAGGGGAACGTCTTCTCATGC
TCCGTGATGCACGAGGCTCTGCACAACCACTACACGCAGA
AGAGCCTCTCCCTGTCTCCGGGTAAATGAGTGCTAGCTGG,
(SEQ ID NO: 34)
GAATTCCAGGGGGTTTAAAATGAATCCAAATCAGAAAATA
ACAACCATTGGATCAATCTGTCTGGTAGTCGGACTAATTA
GCCTAATATTGCAAATAGGGAATATAATCTCAATATGGAT
TAGCCATTCAATTCAAACTGGAAGTCAAAACCATACTGGA
ATATGCAACCAAAACATCATTACCTATAAAAATAGCACCT
GGGTAAAGGACACAACTTCAGTGATATTAACCGGCAATTC
ATCTCTTTGTCCCATCCGTGGGTGGGCTATATACAGCAAA
GACAATAGCATAAGAATTGGTTCCAAAGGAGACGTTTTTA
GATCTGACAAAACTCACACATGCCCACCGTGCCCAGCACC
TGAACTCCTGGGGGGACCGTCAGTCTTCCTCTTCCCCCCA
AAACCCAAGGACACCCTCATGATCTCCCGGACCCCTGAGG
TCACATGCGTGGTGGTGGACGTGAGCCACGAAGACCCTGA
GGTCAAGTTCAACTGGTACGTGGACGGCGTGGAGGTGCAT
AATGCCAAGACAAAGCCGCGGGAGGAGCAGTACAACAGCA
CGTACCGTGTGGTCAGCGTCCTCACCGTCCTGCACCAGGA
CTGGCTGAATGGCAAGGAGTACAAGTGCAAGGTCTCCAAC
AAAGCCCTCCCAGCCCCCATCGAGAAAACCATCTCCAAAG
CCAAAGGGCAGCCCCGAGAACCACAGGTGTACACCCTGCC
CCCATCCCGGGAGGAGATGACCAAGAACCAGGTCAGCCTG
ACCTGCCTGGTCAAAGGCTTCTATCCCAGCGACATCGCCG
TGGAGTGGGAGAGCAATGGGCAGCCGGAGAACAACTACAA
GACCACGCCTCCCGTGCTGGACTCCGACGGCTCCTTCTTC
CTCTACAGCAAGCTCACCGTGGACAAGAGCAGGTGGCAGC
AGGGGAACGTCTTCTCATGCTCCGTGATGCACGAGGCTCT
GCACAACCACTACACGCAGAAGAGCCTCTCCCTGTCTCCG
GGTAAATGAGTGCTAGCTGG,
or
(SEQ ID NO: 35)
GAATTCCAGGGGGTTTAAAATGAATCCAAATCAGAAAATA
ACAACCATTGGATCAATCTGTCTGGTAGTCGGACTAATTA
GCCTAATATTGCAAATAGGGAATATAATCTCAATATGGAT
TAGCCATTCAATTCAAACTGGAAGTCAAAACCATACTGGA
ATATGCAACCAAAACATCATTACCTATAAAAATAGCACCT
GGGTAAAGGACACAACTTCAGTGATATTAACCGGCAATTC
ATCTCTTTGTCCCATCCGTGGGTGGGCTATATACAGCAAA
GACAATAGCATAAGAATTGGTTCCAAAGGAGACGTTTTTG
TCATAAGAGAGCCCTTTATTTCATGTTCTCACTTGGAATG
CAGGACCTTTTTTCTGACCAGATCTGACAAAACTCACACA
TGCCCACCGTGCCCAGCACCTGAACTCCTGGGGGGACCGT
CAGTCTTCCTCTTCCCCCCAAAACCCAAGGACACCCTCAT
GATCTCCCGGACCCCTGAGGTCACATGCGTGGTGGTGGAC
GTGAGCCACGAAGACCCTGAGGTCAAGTTCAACTGGTACG
TGGACGGCGTGGAGGTGCATAATGCCAAGACAAAGCCGCG
GGAGGAGCAGTACAACAGCACGTACCGTGTGGTCAGCGTC
CTCACCGTCCTGCACCAGGACTGGCTGAATGGCAAGGAGT
ACAAGTGCAAGGTCTCCAACAAAGCCCTCCCAGCCCCCAT
CGAGAAAACCATCTCCAAAGCCAAAGGGCAGCCCCGAGAA
CCACAGGTGTACACCCTGCCCCCATCCCGGGAGGAGATGA
CCAAGAACCAGGTCAGCCTGACCTGCCTGGTCAAAGGCTT
CTATCCCAGCGACATCGCCGTGGAGTGGGAGAGCAATGGG
CAGCCGGAGAACAACTACAAGACCACGCCTCCCGTGCTGG
ACTCCGACGGCTCCTTCTTCCTCTACAGCAAGCTCACCGT
GGACAAGAGCAGGTGGCAGCAGGGGAACGTCTTCTCATGC
TCCGTGATGCACGAGGCTCTGCACAACCACTACACGCAGA
AGAGCCTCTCCCTGTCTCCGGGTAAATGAGTGCTAGCTGG.

The present disclosure also contemplates a nucleic acid encoding any fusion protein as disclosed herein, a vector comprising such a nucleic acid of claim, and a cell comprising such a vector. Vectors and cells containing such vectors can be prepared using methods known in the art.

### II. Engineered Feeder Cells, Engineered Plasma Membrane Particles and Engineered Exosomes Comprising Membrane Bound Fc

Compositions according to the disclosure include compositions comprising Fc-bound feeder cells (FCs), compositions comprising Fc-bound engineered plasma membrane (PM) particles, and compositions comprising Fc-bound engineered exosomes. Fc-bound engineered PM particles include PM nanoparticles derived from Fc-bound feeder cells. Fc bound engineered exosomes included exosomes or other extracellular vesicles derived from Fc-bound feeder cells, as also described in further detail below. Alternatively, exosomes may be derived from other sources such as platelets and megakaryocytes.

As used herein, the term “Fc-bound” shall be understood as referring to the coupling of an Fc domain in an inverted orientation (i.e., the amino terminal end facing intracellularly) to the external surface of a feeder cell or engineered particle via a transmembrane peptide. This can be achieved using the Fc fusion peptides disclosed herein. Thus, one aspect of the present disclosure provides a feeder cell composition comprising at least one Fc-bound feeder cell, i.e., a feeder cell comprising an Fc domain bound to an external surface of the feeder cell, as described in further detail below. For example, a feeder cell can be genetically modified to express an Fc domain bound to an external surface of the feeder cell, i.e., to express an Fc fusion peptide as described further below. Another aspect of the disclosure provides an NK cell expanding composition free of feeder cells, comprising at least one Fc-bound engineered particle, i.e., an engineered particle comprising an Fc domain bound in inverted orientation to an external surface of the feeder cell. In some aspects, the feeder cells can be engineered to express an agonist (e.g., agnositic antibody) or ligand that can be tagged with a humanized antibody (such as, for example CD20). In some embodiments, the feeder cell is enginerred to express CD20 and opsonized with Rituxan.

In a feeder cell composition, the at least one Fc-bound feeder cell optionally comprises at least one γδ T cell effector agent, wherein the at least one γδ T cell effector agent comprises a cytokine, an adhesion molecule, or a γδ T cell activating agent. In one example, an Fc-bound feeder cell comprises at least one γδ T cell effector selected from the group consisting of 4-1BBL; CD80; CD86; MICA; UBLP; 2B4; LFA-1; agonist (e.g., agnositic antibody) or ligand for NKG2D, NKp46, NKp44, NKp30, or DNAM-1; agonist (e.g., agnositic antibody) or ligand for Notch, BCM/SLAMF2 or TLR; IL-2; IL-12; IL-18; IL-15; or IL-21; or any combination thereof. In one example, the at least one γδ T cell effector agent comprises 4-1BBL, IL-18, IL-15, or IL-21, or any combination thereof. In one example, an Fc-bound feeder cell comprises one γδ T cell effector which is IL-15 or IL-21. In one example, an Fc-bound feeder cells can comprise at least two or more different γδ T cell effector agents. In one example, an Fc-bound feeder cell comprises at least one γδ T cell effector selected from IL-2, IL-21, or 4-1BBL, or a combination thereof (such as, for example, 4-1BBL and IL-21; 4-1BBL and IL-2; IL-21 and IL-2; or 4-1BBL, IL-2, and IL-21), including, but not limited to membrane bound 4-1BBL, IL-2, or IL-21 or combinations thereof (such as, for example, mb4-1BBL and mbIL-21; mb4-1BBL and mbIL-2; mbIL-2 and mbIL-21; or mb4-1BBL, mbIL-2, and mbIL-21) as well as combinations of membrane bound and non-bound effector agents.

In an γδ T cell expanding composition free of feeder cells, Fc-bound engineered plasma membrane particles optionally comprise at least one cell y6T cell effector agent, wherein the at least one γδ T cell effector agent comprises a cytokine, an adhesion molecule, or a γδ T cell activating agent. In one example, an Fc-bound engineered particle comprises at least one γδ T cell effector selected from the group consisting of 4-1BBL; CD80; CD86; MICA; UBLP; 2B4; LFA-1; agonist (e.g., agnositic antibody) or ligand for NKG2D, NKp46, NKp44, NKp30, or DNAM-1; agonist (e.g., agnositic antibody) or ligand for Notch, BCM/SLAMF2 or TLR; IL-2; IL-12; IL-18; IL-15; or IL-21; or any combination thereof. In one example, the at least one γδ T cell effector agent comprises 4-1BBL, IL-18, IL-15, or IL-21, or any combination thereof. In one example, an Fc-bound engineered particle comprises one cell γδ T cell effector which is IL-15 or IL-21. Fc-bound engineered PM particles can comprise at least two or more different γδ T cell effector agents. In one example, an Fc-bound engineered particle comprises at least one γδ T cell effector selected from IL-2, IL-21, or 4-1BBL, or a combination thereof (such as, for example, 4-1BBL and IL-21; 4-1BBL and IL-2; IL-21 and IL-2; or 4-1BBL, IL-2, and IL-21), including, but not limited to membrane bound 4-1BBL, IL-2, or IL-21 or combinations thereof (such as, for example, mb4-1BBL and mbIL-21; mb4-1BBL and mbIL-2; mbIL-2 and mbIL-21; or mb4-1BBL, mbIL-2, and mbIL-21) as well as combinations of membrane bound and non-bound effector agent.

### (a) Fc-Bound Feeder Cells

The present disclosure provides feeder cells comprising an Fc fusion peptide as detailed above. γδ T cell feeder cells for use in the methods disclosed herein, and for use in making the PM particles and exosomes disclosed herein, can be either irradiated autologous or allogeneic peripheral blood mononuclear cells (PBMCs), fibroblast, epithelial cells, endothelial cells, antigen-presenting cells (e.g., dendritic cells, B cells, mast cells, macrophages, monocytes), T cells, NK cells, a microbial cell or nonirradiated autologous or allogeneic PBMCs, RPMI8866, HFWT, 721.221 or K562 cells as well as EBV-LCLs, other non-HLA or low-HLA expressing cell lines or patient derived primary tumors which can be used as a tumor vaccine. The microbial cells can be a bacterial cell. The microial cells can be those do not cause disease (e.g. Bbacillus Calmette-Guérin). In some embodiments, the microbial cell is a cell of a probiotic (for example, Lactobacillus, Bifidobacterium, Streptococcus, Bacillus, Lactococcus, Enterococcus, Pediococcus, Propionibacterium, Peptostreptococcus, or Saccharomyces). Fc-bound feeder cells can be prepared by transfecting or transducing feeder cells with any Fc fusion peptide as described herein, using standard transduction or transfection techniques well known in the art. For example, cDNA vectors for Fc fusion peptides disclosed herein can be ligated into an expression plasmid, which allows expression in bacterial (E. coli), insect, or mammalian cells. The cDNA vector can be FLAG- or HIS-tagged. Suitable transfection methods include nucleofection (or electroporation), calcium phosphate-mediated transfection, cationic polymer transfection (e.g., DEAE-dextran or polyethylenimine), viral transduction, virosome transfection, virion transfection, liposome transfection, cationic liposome transfection, immunoliposome transfection, nonliposomal lipid transfection, dendrimer transfection, heat shock transfection, magnetofection, lipofection, gene gun delivery, impalefection, sonoporation, optical transfection, and proprietary agent-enhanced uptake of nucleic acids. Transfection methods are well known in the art (see, e.g., “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, NY, 3rd edition, 2001). Alternatively, molecules can be introduced into a cell by microinjection. For example, molecules can be injected into the cytoplasm or nuclei of the cells of interest. The amount of each molecule introduced into the cell can vary, but those skilled in the art are familiar with means for determining the appropriate amount.

In one example, the feeder cell used in this invention may be a peripheral blood mononuclear cell (PBMC), a fibroblast, an epithelial cell, an endothelial cell, an antigen-presenting cell, a microbial cell, or a cell line, wherein the cell line may be RPMI8866, HFWT, 721.221, 1(562, or EBV-LCL.

It will be understood that various molecules can be introduced into a cell simultaneously or sequentially. For example, an Fc fusion peptide and one or more membrane bound γδ T cell effector agents can be introduced to a feeder cell at the same time. Alternatively, one can be introduced first and then the other molecule(s) can later be introduced into the cell. For example, feeder cells once having been transfected or transduced with an Fc fusion peptide can be further transfected with membrane bound γδ T cell effector agents such as IL-2, IL-15 and/or IL-21 and/or 41BBL and/or infected as an EBV-LCL and/or other γδ T cell effector agent(s). Alternatively, feeder cells can be simultaneously transfected or transduced with an Fc fusion peptide and membrane bound γδ T cell effector agents such as IL-2, IL-15 and/or IL-21 and/or 41BBL and/or EBV-LCL and/or other γδ T cell effector agent(s). Alternatively, feeder cells previously transfected or transduced and expressing membrane bound γδ T cell effector agents such as IL-2, IL-15 and/or IL-21 and/or 41BBL and/or infected as an EBV-LCL and/or other γδ T cell effector agent(s), can be transfected or transduced with an Fc fusion peptide. It will be also appreciated that other means such as chemical conjugation methods known in the art can be used to achieve a membrane bound Fc.

In general, apart from the contact with the compositions disclosed herein, the cell is maintained under conditions appropriate for cell growth and/or maintenance. Suitable cell culture conditions are well known in the art and are described, for example, in Santiago et al., Proc. Natl. Acad. Sci. USA, 2008, 105:5809-5814; Moehle et al. Proc. Natl. Acad. Sci. USA, 2007, 104:3055-3060; Urnov et al., Nature, 2005, 435:646-651; and Lombardo et al., Nat. Biotechnol., 2007, 25:1298-1306. Those of skill in the art appreciate that methods for culturing cells are known in the art and can and will vary depending on the cell type. Routine optimization may be used, in all cases, to determine the best techniques for a particular cell type.

Fc-bound feeder cells can be used in cell culture to stimulate γδ T cells directly or can be used to prepare plasma membrane particles or exosomes derived from the feeder cells.

### (b) Fc-Bound Engineered Plasma Particles

Fc-bound engineered PM (plasma membrane) particles include Fc-bound PM particles, which can be prepared from Fc-bound γδ T cell feeder cells using well known methods. PM particles are vesicles made from the plasma membrane of a cell or artificially made (i.e., liposomes). A PM particle can contain a lipid bilayer or simply a single layer of lipids. A PM particle can be prepared in single lamellar, multi-lamellar, or inverted form. PM particles can be prepared from Fc-bound feeder cells as described herein, using known plasma membrane preparation protocols or protocols for preparing liposomes such as those described in U.S. Pat. No. 9,623,082, the entire disclosure of which is herein incorporated by reference. In certain aspects, PM particles as disclosed herein range in average diameter from about 170 to about 300 nm.

### (c) Fc-Bound Engineered Plasma Particles

Fc-bound exosomes as disclosed herein can be prepared from exosome-secreting cells, which can be prepared from Fc-bound feeder cells using well known methods, wherein the exosome is an extracellular product of exosome-secreting cells, as described in United States Pat. App. Pub. No. 20170333479, the entire disclosure of which is herein incorporated by reference. Exosomes comprise lipids and proteins and the identity of the proteins found in a particular exosome is dependent on the cell(s) that produced them. Exosomes disclosed herein comprise an Fc fusion peptide as disclosed herein (i.e., are Fc-bound), and optionally one or more stimulatory peptides (γδ T cell effector agents) present in the exosome membrane. Exosomes can be produced for example from cell lines engineered for improved formation or release of exosomes. Such cell lines include, but are not limited to, Fc-bound cell lines as described above in Section II(a). Non-limiting cell lines are Fc-bound K562-mb15-41BBL and Fc-bound K562-mb21-41BBL. In certain aspects, exosomes as disclosed herein range in average diameter from about 30 to about 100 nm, or to about 160 nm. In one aspect, exosomes average about 60-80 nm in diameter. The ability with exosomes to achieve particle sizes smaller than readily achieved with PM particles means that exosomes can be more readily adapted to uses where a smaller size is preferable. For example, exosomes may be preferred in applications requiring diffusion through physiological barriers, enhanced biodistribution through tissue compartments, or intravenous injections.

### III. Compositions.

The present disclosure provides various γδ T cell expanding compositions comprising Fc-bound feeder cells as disclosed above, and in other aspects, γδ T cell expanding compositions free of feeder cells, comprising one or more engineered Fc-bound particles such as PM particles or exosomes as disclosed above. Any of the Fc-bound feeder cells or Fc-bound engineered PM particles used in the compositions optionally further comprise at least one, two, or more different γδ T cell effector agents. In one aspect, one γδ T cell effector agent is IL-21, and in some aspects, one γδ T cell effector agent is IL-21 and a second is 4-1BBL. The Fc-bound feeder cells or Fc-bound engineered PM particles optionally comprise one or more additional γδ T cell effector agents as disclosed above.

A γδ T cell expanding composition that comprises a PM particle comprising a plasma membrane, may further comprise a plurality of microparticles/nanoparticles, wherein the plasma membrane coats the plurality of microparticles. Microparticles/nanoparticles can comprise magnetic microparticles, silica beads, polystyrene beads, latex beads, a particulate contrast agent, a particulate cancer therapeutic agent, or any combination thereof.

The present disclosure also contemplates a γδ T cell expanding infusion formulation comprising any of the γδ T cell expanding compositions disclosed herein, combined with a pharmaceutically acceptable carrier.

Therapeutic, pharmaceutical compositions can be prepared by combining the Fc-bound feeder cells or engineered PM particles disclosed herein with a pharmaceutically acceptable carrier as known in the art, as described for example in Remington: The Science and Practice of Pharmacy (19th ed.) ed. A. R. Gennaro, Mack Publishing Company, Easton, Pa. 1995. Examples of pharmaceutically acceptable carriers include, but are not limited to: sterile water, saline, Ringer's solution, dextrose solution, and buffered solutions at physiological pH. For example, the pH of the solution is preferably from about 5 to about 8, and more preferably from about 7 to about 7.5.

It will be apparent to those persons skilled in the art that certain carriers can be more preferable depending upon, for instance, the route of administration and concentration of composition being administered. The pharmaceutical composition can be suitably prepared for administration via any of a number of known routes of administration to mammals, especially humans, depending on whether local or systemic treatment is desired, and on the area to be treated. Administration can be topical (including ophthalmic, vaginal, rectal, intranasal), oral, by inhalation, or parenteral, for example by intravenous drip or injection, or subcutaneous, intraperitoneal, intramuscular, intracavity, or transdermal injection.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like can be necessary or desirable.

Some of the compositions can potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

An γδ T cell expanding infusion formulation can thus be formulated for parenteral infusion, arterial infusion, venous infusion, artificial catheter mediated infusion, intravenous, intraperitoneal, subcutaneous injection, oral or topical delivery. In some embodiments, the method of any preceding aspect comprises administering the γδ T cells parenterally, intravenously, intraperitoneally, or subcutaneously, or through arterial infusion, venous infusion, or artificial catheter mediated infusion.

In one aspect, the present disclosure contemplates any γδ T cell expanding composition prepared in vitro or ex vivo as disclosed herein, administered to or infused into a subject in need of γδ T cell expansion. It is understood and herein contemplated that infusion can occur in vitro with a commercial source of γδ T cells or ex vivo from a donor source (such as, for example an allogeneic donor or autologous donor source (i.e., the recipient subject receiving the expanded γδ T cells).

In another aspect, the present disclosure contemplates a γδ T cell composition comprising an in vitro γδ T cell population in contact with an Fc-bound feeder cell composition as disclosed herein, or a feeder cell free, Fc-bound NK cell expanding composition as disclosed herein.

In another aspect, the present disclosure contemplates an expanded population of γδ T cells exposed in vitro to a γδ T cell expanding composition, the composition being free of feeder cells and comprising at least one Fc-bound engineered particle as disclosed herein, comprising at least two γδ T effector agents, wherein the at least two γδ T cell effector agents are selected from IL-2, IL-21, IL-15, or 4-1BBL, or any combination thereof. In another aspect, the present disclosure contemplates an expanded population of γδ T cells exposed in vitro to a γδ T cell expanding composition, the composition being free of feeder cells and comprising at least one Fc-bound engineered particle as disclosed herein, comprising at least two γδ T cell effector agents, wherein one of the at least two γδ T cell effector agents is IL-21 or 4-1BBL. In one example, the γδ T effector agent is IL-2. In one example, the γδ T effector agent is IL-21. In one example, the γδ T effector agent is IL-15. In one example, the γδ T effector agent is 4-1BBL. The expanded population of γδ T cells can exhibit increased cytotoxicity compared to non-expanded γδ T cells.

In different aspects, the expanded population of γδ T cell can exhibit cytotoxicity of at least about 2×, 5× or 10× that of non-expanded γδ T cells, which can be determined with increased percentages of γδ T cell population producing cytotoxic effectors (e.g., IFNγ, TNFα, perforin, granzymes), or increased production/expression levels of cytotoxic effectors (e.g., IFNγ, TNFα, perforin, granzymes) or increased expression levels of molecules for killing (e.g., FasL, TRAIL).

In some embodiments, the method of any preceding aspect comprises contacting the at least one γδ T cell with the feeder cell, the engineered particle, the exosome, or the solid support in vitro, in vivo, or ex vivo. In some embodiments, the expanded γδ T cells comprise Vδ2 subtype and/or Vδ1 subtype. The the γδ T cells may be autologous, haploidentical, or allogeneic γδ T cells. In some embodiments, the γδ T cells are expanded for at least 14 days, wherein at least about 5%, 10%, 20%, 30%, 40%, 50%, or 60% of the cells in the expanded cells are γδ T-cells of the Vδ2 subtype.

In some embodiments, the γδ T cells expand at a faster rate over 14 days than a control γδ T cell population. It should be understood herein that the term “control γδ T cell population” refers to the γδ T cells prior to contacting to the Fc-bound feeder cells, exosomes, engineered plasma membrane particle, or solid supports disclosed herein, or refers to the γδ T cells contacting the feeder cells, exosomes, engineered plasma membrane particle, or solid supports with the Fc disclosed herein.

The γδ T cells expanded according to the methods of any preceding aspect can be an isolated cell population or in a mixed cell population. The mixed cell population can be depleted of NK cells prior to, during, or after expansion of the γδ T cells. Accordingly, this method can lead to co-expansion of γδ T and NK cells if NK cells are not removed prior to expansion. The combination of these two populations can lead to broader antitumor function and thus better efficacy. Such γδ T cells or γδ T/NK cell mixture can be utilized as therapeutics for treatment of diseases.

In another aspect, the present disclosure provides a composition comprising a therapeutic dose of γδ T cells comprising an expanded population of γδ T cells as disclosed herein, optionally in combination with a pharmaceutically acceptable carrier. The expanded population of γδ T cells can exhibit higher CD16 and other advantageous properties such as higher cytotoxicity. An amount of γδ T cells that provides a therapeutic dose will vary on a number of factors as appreciated by those of skill in the art, and are discussed for example in U.S. Pat. No. 9,907,820, the entire disclosure of which is herein incorporated by reference. Factors include age, gender and diagnosis of the subject, and route of administration, which may be but is not limited to oral, buccal, mucosal, and intravenous routes. For example, suitable doses for a therapeutic effect would be at least 10⁴ or between about 10⁴ and about 10¹⁰ cells per dose, from about 10⁴ to about 10⁸ cells per dose, or from about 10⁵ to about 10⁷ cells per dose, for example, preferably in a series of dosing cycles. An exemplary dosing regimen consists of four one-week dosing cycles of escalating doses, starting at least at about 10⁵ cells on Day 0, for example increasing incrementally up to a target dose of about 10¹⁰ cells within several weeks of initiating an intra-patient dose escalation scheme. Suitable modes of administration include intravenous, subcutaneous, intracavitary (for example by reservoir-access device), intraperitoneal, and direct injection into a tumor mass. It will be appreciated that the equivalent of a therapeutic dose as expressed above can be alternatively expressed in an amount per total body surface area.

The γδ T cell effector or the receptors thereof disclosed herein comprises, 4-1BBL (HGNC: 11939 Entrez Gene: 8744 Ensembl: ENSG00000125657 OMIM: 606182 UniProtKB: P41273), CD80 (HGNC: 1700 Entrez Gene: 941 Ensembl: ENSG00000121594 OMIM: 112203 UniProtKB: P33681), CD86 (HGNC: 1705 Entrez Gene: 942 Ensembl: ENSG00000114013 OMIM: 601020 UniProtKB: P42081), MICA (HGNC: 7090 Entrez Gene: 100507436 Ensembl: ENSG00000204520 OMIM: 600169 UniProtKB: Q29983), UBLP, 2B4 (HGNC: 18171 Entrez Gene: 51744 Ensembl: ENSG00000122223 OMIM: 605554 UniProtKB: Q9BZW8), LFA-1 (CD11a/CD18, CD11a (HGNC: 6148 Entrez Gene: 3683 Ensembl: ENSG00000005844 OMIM: 153370 UniProtKB: P20701) and CD18 (HGNC: 6155 Entrez Gene: 3689 Ensembl: ENSG00000160255 OMIM: 600065 UniProtKB: P05107), ICAM-1 (HGNC: 5344 Entrez Gene: 3383 Ensembl: ENSG00000090339 OMIM: 147840 UniProtKB: P05362), ligand for NKG2D (HGNC: 18788 Entrez Gene: 22914 Ensembl: ENSG00000213809 OMIM: 611817 UniProtKB: P26718), NKp46 (HGNC: 6731 Entrez Gene: 9437 Ensembl: ENSG00000189430 OMIM: 604530 UniProtKB: O76036), NKp44 (HGNC: 6732 Entrez Gene: 9436 Ensembl: ENSG00000096264 OMIM: 604531 UniProtKB: 095944), or NKp30 (HGNC: 19077 Entrez Gene: 259197 Ensembl: ENSG00000204475 OMIM: 611550 UniProtKB: O14931), agonist (e.g., agnositic antibody) or ligand for DNAM-1 (HGNC: 16961 Entrez Gene: 10666 Ensembl: ENSG00000150637 OMIM: 605397 UniProtKB: Q15762), IL-2 (HGNC: 6001 Entrez Gene: 3558 Ensembl: ENSG00000109471 OMIM: 147680 UniProtKB: P60568), IL-12 (HGNC: 5969 Entrez Gene: 3592 Ensembl: ENSG00000168811 OMIM: 161560 UniProtKB: P29459), IL-18 (HGNC: 5986 Entrez Gene: 3606 Ensembl: ENSG00000150782 OMIM: 600953 UniProtKB: Q14116), IL-15 (HGNC: 5977 Entrez Gene: 3600 Ensembl: ENSG00000164136 OMIM: 600554 UniProtKB: P40933), IL-21 (HGNC: 6005 Entrez Gene: 59067 Ensembl: ENSG00000138684 OMIM: 605384 UniProtKB: Q9HBE4), CD69 (HGNC: 1694 Entrez Gene: 969 Ensembl: ENSG00000110848 OMIM: 107273 UniProtKB: Q07108), CD25 (HGNC: 6008 Entrez Gene: 3559 Ensembl: ENSG00000134460 OMIM: 147730 UniProtKB: P01589), RANKL (HGNC: 11926 Entrez Gene: 8600 Ensembl: ENSG00000120659 OMIM: 602642 UniProtKB: O14788).

### IV. Methods and Uses

### (a) Methods and Uses for Increasing Cytotoxicity of γδ T Cells

In one aspect, the present disclosure provides a method for increasing γδ T cell cytotoxicity, by expanding an initial population of γδ T cells using a γδ T cell expanding composition or formulation as disclosed herein. Alternatively, the present disclosure provides a use of a γδ T cell expanding composition or formulation as disclosed herein, for increasing γδ T cell cytotoxicity, by expanding an initial population of γδ T cells. The disclosed methods and uses provide a simple expansion platform which avoids a complicated alternative process for expansion involving for example, coating a solid support with monoclonal antibody, and using soluble cytokine(s) in solution. Instead, in the methods and uses disclosed herein, an initial population of γδ T cells is obtained from a donor, and exposed to a γδ T cell expanding composition as disclosed herein. In therapeutic methods, exposure can be in vitro or in vivo. In uses, exposure can be in vitro or ex vivo. In any of the methods and uses, γδ T cells are contacted with one or more Fc-bound feeder cells, Fc-bound PM particles or Fc-bound exosomes or any combination thereof. The exposed Fc domain binds to CD16 on the surface of the γδ T cells resulting in stimulation of the γδ T cells to expand faster and/or more efficiently, and to produce γδ T cells with higher anti-tumor toxicity and γδ T cells with a more favorable overall phenotype.

In any of the methods or uses, the composition in contact with the γδ T cells can comprise any of the Fc-bound feeder cells or Fc-bound engineered PM particles or Fc-bound engineered exosome disclosed herein. Engineered PM particles can be Fc-bound PM particles. In one aspect, an optionally present γδ T cell effector agent is IL-21 or IL-15. An optionally present second γδ T cell effector agent can be selected from 4-1BBL, IL-2, IL-12, IL-15, IL-18, IL-21, MICA, UBLP, 2B4, LFA-1, a Notch ligand, agonists (e.g., agnositic antibody) or ligands for NKp46, or BCM1/SLAMF2, agonists (e.g., agnositic antibody) or ligands for TLR and NKG2D. In one aspect, a second NK cell effector agent is 4-1BBL. The composition can further comprise at least one additional (i.e., a third, fourth, fifth, etc.) γδ T cell effector agent selected from IL-2, IL-12, IL-15, IL-18, IL-21, MICA, UBLP, 2B4, LFA-1, a Notch ligand, agonists (e.g., agnositic antibody) or ligands for NKp46, or BCM1/SLAMF2, agonists (e.g., agnositic antibody) or ligands for TLR and NKG2D. In some embodiments, the γδ T cell effector agent comprises 4-1BBL, IL-18, IL-15, or IL-21, or any combination thereof γδ T cell expansion performed in this way can achieve much greater than several (about 3-4 folds) in 10 days. Rather, γδ T cell expansion according to the present methods can achieve at least about 100 fold, about 200 fold, about 300 fold, about 400 fold, about 500 fold, about 600 fold, about 700 fold, about 800 fold, about 900 fold, about 1100 fold, about 1200 fold, about 1300 fold, about 1400 fold, about 1500 fold, about 1600 fold, about 1700 fold, about 1800 fold, about 1900 fold up, to about 2000 fold increase in γδ T cell numbers in 2, 3, 4, 5, 6, 7, 8, 9, 10, 11, 12, 13, 14, 15, 16, 17, 18, 19, or 20, days, 3 weeks, 4 weeks, 5 weeks, or 6 weeks, or greater with longer time. Thus, the disclosed methods and uses are useful for scaled-up manufacturing of γδ T cells. Sources of γδ T cells may be from peripheral blood, splenic γδ T cells, lymphocyte preparations such as buffy coats, iPSC derived γδ T cells, ESC derived γδ T cells, and genetically modified/engineered γδ T cells, or any genetically modified γδ T cells, including but not limited to γδ T cells derived from polymorphisms of the Fc receptor, such as a Phe or Val at position 158, such as those known in the art and described for example in Blood (1997) 90:1109-14, and J Clin Invest. (1997) 100:1059-70. Such genetically modified γδ T cell sources can be engineered using methods known in the art. Alternatively, γδ T cells can be derived from a cell donor that carries a desired polymorphism and the donated cells used as an initial population of γδ T cells that are expanded by the methods and using the composition described herein. Thus, in this context “genetically modified” encompasses naturally occurring γδ T cells carrying a polymorphism. The method may be applied to γδ T cells from human origin or other animals.

In some embodiments, the method or use of any preceding aspect comprises contacting the at least one γδ T cell with the feeder cell, the engineered particle, the exosome, or the solid support in vitro, in vivo, or ex vivo. In some embodiments, the expanded γδ T cells comprise Vδ2 subtype and/or Vδ1 subtype. The the γδ T cells may be autologous, haploidentical, or allogeneic γδ T cells. In some embodiments, the γδ T cells are expanded for at least 14 days, wherein at least about 5%, 10%, 20%, 30%, 40%, 50%, or 60% of the cells in the expanded cells are γδ T-cells of the V62 subtype.

In some embodiments, the γδ T cells expand at a faster rate over 14 days than a control γδ T cell population. It should be understood herein that the term “control γδ T cell population” refers to the γδ T cells prior to contacting to the Fc-bound feeder cells, exosomes, engineered plasma membrane particle, or solid supports disclosed herein, or refers to the γδ T cells contacting the feeder cells, exosomes, engineered plasma membrane particle, or solid supports with the Fc disclosed herein.

The γδ T cells expanded according to the methods or uses of any preceding aspect can be an isolated cell population or in a mixed cell population. The mixed cell population can be depleted of NK cells prior to, during, or after expansion of the γδ T cells. Accordingly, this method can lead to co-expansion of γδ T and NK cells if NK cells are not removed prior to expansion. The combination of these two populations can lead to broader antitumor function and thus better efficacy. Such γδ T cells or γδ T /NK cell mixture can be utilized as therapeutics for treatment of diseases.

Moreover, the disclosed methods and uses have the added benefit of providing cells with higher cytotoxicity. An initial population of γδ T cells expanded according to the disclosed methods produces an expanded population of γδ T cells that exhibits at least about 2× the cytotoxicity of the initial population of γδ T cells, at least about 4× the cytotoxicity of the initial population of γδ T cells, at least about 5× that of the initial population of γδ T cells, at least about 8× the cytotoxicity of the initial population of γδ T cells, or at least about 10× that of the initial population of γδ T cells. Higher expression of ADCC-related proteins such as, in non-limiting example, CD16; or other γδ T cell ligands such as, in non-limiting example, NKG2D, NKp46, CD62L, ICAM-1 can be used to assess relative cytotoxicity of expanded γδ T cells as compared to non-expanded γδ T cells or γδ T cells expanded under other conditions. Markers such as CD69, CD25, and RANKL, etc., are indicators of γδ T cells in an activated state. In combination the markers can provide a signal of increased cytotoxicity, even when cytotoxicity cannot be assessed directly. For example, an expanded population of γδ T cells as disclosed herein can exhibit increased killing of tumor targets or secrete higher amounts of anti-tumor or anti-pathogen cytokines (e.g., IFNγ, TNFα, perforin, granzymes) or express increased levels of molecules for killing (e.g., FasL, TRAIL) compared with non-expanded γδ T cells. In another aspect, an expanded population of γδ T cells as disclosed herein can exhibit increased expression of CD69, CD25, NKG2D, NKp46 and/or CD16 compared with non-expanded γδ T cells. Various means for detecting amounts of a specific protein to assess the activation state of γδ T cells are known in the art and can be used, including spectrometry methods such as flow cytometry or immunodetection methods such as Western blot, Enzyme-linked immunosorbent assay (ELISA), protein immunoprecipitation; immunoelectrophoresis, or immunostaining.

Additionally, an expanded population of γδ T cells as disclosed herein can exhibit improved ability to withstand cryopreservation, retaining viability and cytotoxicity and following freeze and thaw.

### (b) Therapeutic Methods and Uses

The compositions and methods disclosed herein can be used in a variety of therapeutic, diagnostic, industrial, and research applications. In some aspects, the present disclosure can be used to treat cancer. Accordingly, in one aspect, disclosed herein are methods of treating, inhibiting, reducing, and/or preventing a cancer, cancer recurrence, or metastasis or an infectious disease such as a viral infection or bacterial infection in a subject comprising administering to the subject in need thereof an effective amount of a composition or an expanded γδ T cell population as described herein.

Accordingly, in some aspects, disclosed herein is a is a method of treating, decreasing, inhibiting, reducing, ameliorating, and/or preventing a cancer, metastasis, or an infectious disease in a subject comprising
a. obtaining at least one γδ T cell;
b. contacting the least one γδ T cell with an engineered feeder cell, an engineered plasma membrane particle, an exosome, or a solid support comprising a Fc domain bound to the external surface thereof;
c. administering to the subject a therapeutically effective amount of the contacted γδ T cells to the subject.

In some embodiments, step b further comprises inducing, activating, and/or expanding the at least one γδ T cell following the contact with the engineered feeder cell, the engineered plasma membrane particle, the exosome, or the solid support comprising a Fc domain bound to the external surface thereof, wherein the γδ T cells are induced, activated, and/or expanded for at least 14 days.

In some embodiments, the engineered feeder cell, engineered plasma membrane particle, the exosome, or the solid support may further comprise at least one γδ T cell effector agent, wherein the at least one γδ T cell effector agent comprises 4-1BBL; CD80; CD86; MICA; UBLP; 2B4; LFA-1; agonist (e.g., agnositic antibody) or ligand for NKG2D, NKp46, NKp44, NKp30, or DNAM-1; agonist (e.g., agnositic antibody) or ligand for Notch, BCM/SLAMF2 or TLR; IL-2; IL-12; IL-18; IL-15; or IL-21; or any combination thereof. In some embodiments, the at least one γδ T cell effector agent comprises 4-1BBL, IL-18, IL-15, or IL-21, or any combination thereof.

In some aspects, disclose herein is a method of treating, decreasing, inhibiting, reducing, ameliorating, and/or preventing a cancer, metastasis, or an infectious disease in a subject by expanding, inducing, and/or activating endogenous γδ T cells in the subject, said method comprising administering to the subject an engineered plasma membrane particle, an exosome, or a solid support comprising a Fc domain bound to the external surface thereof, wherein the engineered feeder cell, engineered plasma membrane particle, the exosome, or the solid support may further comprise at least one γδ T cell effector agent, wherein the at least one γδ T cell effector agent comprises 4-1BBL; CD80; CD86; MICA; UBLP; 2B4; LFA-1; agonist (e.g., agnositic antibody) or ligand for NKG2D, NKp46, NKp44, NKp30, or DNAM-1; agonist (e.g., agnositic antibody) or ligand for Notch, BCM/SLAMF2 or TLR; IL-2; IL-12; IL-18; IL-15; or IL-21; or any combination thereof. In some embodiments, the at least one γδ T cell effector agent comprises 4-1BBL, IL-18, IL-15, or IL-21, or any combination thereof.

In some embodiments, the methods of any preceding aspect further comprising administering to the subject an ex vivo composition comprising a fusion protein comprising a transmembrane domain linked to the amino terminus of an Fc domain and bound to an engineered feeder cell, an engineered plasma membrane particle, an exosome, or a solid support, in contact with an isolated mixed cell population comprising at least one γδ T cells comprising CD16 or a functional fragment thereof. In some embodiments, the ex vivo composition further comprises at least one γδ T cell effector agent, wherein the at least one γδ T cell effector agent comprises 4-1BBL; CD80; CD86; MICA; UBLP; 2B4; LFA-1; agonist (e.g., agnositic antibody) or ligand for NKG2D, NKp46, NKp44, NKp30, or DNAM-1; agonist (e.g., agnositic antibody) or ligand for Notch, BCM/SLAMF2 or TLR; IL-2; IL-12; IL-18; IL-15; or IL-21; or any combination thereof. In some embodiments, the at least one γδ T cell effector agent comprises 4-1BBL, IL-18, IL-15, or IL-21, or any combination thereof. The engineered plasma membrane particle can comprise a plasma membrane and a plurality of microparticles or support surfaces, wherein the plasma membrane coats the plurality of microparticles or support surfaces. In some embodiments, the plurality of microparticles or surfaces comprise at least one of magnetic microparticles, silica beads, polystyrene beads, latex beads, micro-structures, a contrast agent, and a cancer therapeutic agent.

A cancer can be selected from, but is not limited to, a hematologic cancer, lymphoma, colorectal cancer, colon cancer, lung cancer, a head and neck cancer, ovarian cancer, prostate cancer, testicular cancer, renal cancer, skin cancer, cervical cancer, pancreatic cancer, and breast cancer. In one aspect, the cancer comprises a solid tumor. In another aspect, the cancer is selected from acute myeloid leukemia, myelodysplastic syndrome, chronic myeloid leukemia, acute lymphoblastic leukemia, myelofibrosis, multiple myeloma. In another aspect, the cancer is selected from a leukemia, a lymphoma, a sarcoma, a carcinoma and may originate in the marrow, brain, lung, breast, pancreas, liver, head and neck, skin, reproductive tract, prostate, colon, liver, kidney, intraperitoneum, bone, joint, eye.

Any of the disclosed treatment methods may further comprise administering to the subject (concurrently, simultaneously, or as a singular formulation) an additional therapeutic agent or regimen in combination with the effective amount of a composition or an expanded γδ T cell population as described herein. An additional therapeutic agent can be a drug-based preparative regimen such as Cy-Flu, Bu-Flu, Flu-Mel or similar with adjustments in dosage or dosing. Alternatively, the additional therapeutic agents or regimens can be selected from chemotherapy agents and regimens such as, in non-limiting example, those known by the acronyms CHOP, FLAG (including FLAG-Ida or FLAG-IDA or IDA-FLAG or Ida-FLAG; and FLAG-Mito or FLAG-MITO or Mito-FLAG or MITO-FLAG or FLANG), IA or IAC, or 7+3. Alternatively, an effective amount of any of the disclosed compositions and/or an expanded γδ T cell population as described herein. may be used in the treatment of any the diseases as described herein, optionally concurrently, simultaneously, or as a singular formulation in combination with the use of an additional therapeutic agent or regimen. In such uses, an additional therapeutic agent can be a drug-based preparative regimen such as Cy-Flu, Bu-Flu, Flu-Mel or similar with adjustments in dosage or dosing. Alternatively, the additional therapeutic agents or regimens can be selected from chemotherapy agents and regimens such as, in non-limiting example, those known by the acronyms CHOP, FLAG (including FLAG-Ida or FLAG-IDA or IDA-FLAG or Ida-FLAG; and FLAG-Mito or FLAG-MITO or Mito-FLAG or MITO-FLAG or FLANG), IA or IAC, or 7+3.

For example, it is intended herein that the disclosed methods of inhibiting, reducing, and/or preventing cancer metastasis and/or recurrence can comprise the administration of any anti-cancer agent known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane),Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPDX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). Also contemplated herein are chemotherapeutics that are PD1/PDL1 blockade inhibitors (such as, for example, lambrolizumab, nivolumab, pembrolizumab, pidilizumab, BMS-936559, Atezolizumab, Durvalumab, or Avelumab). It is also intended herein that the disclosed uses of the disclosed compositions and/or an expanded γδ T cell population for inhibiting, reducing, and/or preventing cancer metastasis and/or recurrence can comprise use in combination the use of any anti-cancer agent known in the art including, but not limited to those agents listed above.

In some aspects, the therapeutic methods and uses of the compositions all as disclosed herein are for treating an infectious disease caused by a viral infection, wherein the viral infection comprises an infection of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papillomavirus, Respiratory syncytial virus, Adenovirus, Coxsackie virus, Dengue virus, Mumps virus, Poliovirus, Rabies virus, Rous sarcoma virus, Reovirus, Yellow fever virus, Zika virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, or Human Immunodeficiency virus type-2.

Alternatively, in any of the therapuetic methods or uses for treatment, the additional therapeutic agent can be an antiviral agent selected from but not limited to a 5-substituted 2-deoxyuridine analog, a nucleoside analogs, a (nonnucleoside) pyrophosphate analog, a nucleoside reverse transcriptase (RT) inhibitors (NRTI), a nonnucleoside reverse transcriptase inhibitor (NNRTI), a protease inhibitor (PI), and integrase inhibitor, an entry inhibitor, and acyclic guanosine analog, an acyclic nucleoside phosphonate (ANP) analog, a hepatitis C virus (HCV) NS5A and NS5B inhibitor, and influenza virus inhibitor, an immunostimulator, an interferon, an oligonucleotide, and an antimitotic inhibitor. Non-limiting examples of antiviral agents are acyclovir, famciclovir, valacyclovir, penciclovir, ganciclovir, ritonavir, lopinavir, saquinavir, and the like; cimetidine; ranitidine; captopril; metformin; bupropion; fexofenadine; oxcarbazepine; leveteracetam; tramadol; or any of their isomers tautomers, analogs, polymorphs, solvates, derivatives, or pharmaceutically acceptable salts.

In some aspects, the methods and uses of the compositions all as disclosed herein are for treating infectious disease caused by a bacterial infection, wherein the bacterial infection comprises an infection of Mycobaterium tuberculosis, Mycobaterium bovis, Mycobaterium bovis strain BCG, BCG substrains, Mycobaterium avium, Mycobaterium intracellular, Mycobaterium africanum, Mycobaterium kansasii, Mycobaterium marinum, Mycobaterium ulcerans, Mycobaterium avium subspecies paratuberculosis, Nocardia asteroides, other Nocardia species, Legionella pneumophila, other Legionella species, Acetinobacter baumanii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii, other Bordetella species, Burkholderia mallei, Burkholderia psuedomallei, Burkholderia cepacian, Chlamydia pneumoniae, Chlamydia trachomatis, Chlamydia psittaci, Coxiella burnetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, Clostridium difficile, other Clostridium species, Yersinia enterolitica, and other Yersinia species, and Mycoplasma species.

In some aspects, the methods and uses of the compositions all as disclosed herein are for treating infectious disease caused by a fungal infection, wherein the fungal infection comprises an infection of Candida albicans, Cryptococcus neoformans, Histoplama capsulatum, Aspergillus fumigatus, Coccidiodes immitis, Paracoccidiodes brasiliensis, Blastomyces dermitidis, Pneumocystis carinii, Penicillium marneffi, or Alternaria alternate.

In some aspects, the methods and uses of the compositions all as disclosed herein are for treating infectious disease caused by a parasitic infection, wherein the parasitic infection comprises an infection of Toxoplasma gondii, Plasmodium falciparum, Plasmodium vivax, Plasmodium malariae, other Plasmodium species, Entamoeba histolytica, Naegleria fowleri, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Diphyllobothrium latum, Clonorchis sinensis; Clonorchis viverrini, Fasciola hepatica, Fasciola gigantica, Dicrocoelium dendriticum, Fasciolopsis buski, Metagonimus yokogawai, Opisthorchis viverrini, Opisthorchis felineus, Clonorchis sinensis, Trichomonas vaginalis, Acanthamoeba species, Schistosoma intercalatum, Schistosoma haematobium, Schistosoma japonicum, Schistosoma mansoni, other Schistosoma species, Trichobilharzia regenti, Trichinella spiralis, Trichinella britovi, Trichinella nelsoni, Trichinella nativa, or Entamoeba histolytica.

Alternatively, in any of the methods or uses the additional therapeutic agent can be an antibiotic agent selected from but not limited to penicillin, tetracycline, cephalosporin, lincomycin, a macrolide, a sulfonamide, a glycopeptide, an aminoglycosides, and a carbapenem. Non-limiting examples of antiviral agents are amoxicillin, doxycycline, cephalexin, ciprofloxacin, clindamycin, metronidazole, azithromycin, sulfamethoxazole and trimethoprim, clavulanate, and levofloxacin.

In some embodiments, the γδ T cells administered or used in the method or uses of any preceding aspect are formulated in a pharmaceutically acceptable carrier and a pharmaceutically acceptable excipient.

As the timing of a cancer, metastatic condition, or infection can often not be predicted, it should be understood the disclosed methods of treating, preventing, reducing, and/or inhibiting a cancer, metastatic condition, or infection, or the use of any of the disclosed compositions or combinations for such treating, preventing, reducing, and/or inhibiting of a cancer, metastatic condition, or infection, can be practiced prior to or following the onset of the cancer, metastatic condition, or infection, to treat, prevent, inhibit, and/or reduce the muscular disease. In one aspect, the disclosed methods or uses can be employed 30, 29, 28, 27, 26, 25, 24, 23, 22, 21, 20, 19, 18, 17, 16, 15, 14, 13, 12, 11, 10, 9, 8, 7, 6, 5, 4, 3 days, 60, 48, 36, 30, 24, 18, 15, 12, 10, 9, 8, 7, 6, 5, 4, 3, 2 hours, 60, 45, 30, 15, 10, 9, 8, 7, 6, 5, 4, 3, 2, or 1 minute prior to a cancer, a metastatic condition, or an infection; concurrently with the cancer, metastatic condition, or infection; or 1, 2, 3, 4, 5, 6, 7, 8, 9, 10, 15, 20, 25, 30, 35, 40, 45, 50, 55, 60, 75, 90, 105, 120 minutes, 3, 4, 5, 6, 7, 8, 9, 10, 11, 12, 15, 18, 24, 30, 36, 48, 60 hours, 3, 4, 5, 6, 7, 8, 9, 10, 11, 12, 13, 14, 15, 16, 17, 18, 19, 20, 21, 22, 23, 24, 25, 26, 27, 28, 29, 30, 45, 60, 90 days, 4, 5, 6, 7, 9, 10, 11, 12 months, 2, 3, 4, 5, 6, 7, 8, 9, 10, 11, 12, 13, 14, 15, 16, 17, 18, 19, 20, 30, 40, 50 years or more post cancer, metastatic condition, or infection.

### V. Kit.

A further aspect of the present disclosure provides kits comprising the at least one of the fusion peptides as detailed above, and/or at least one of the Fc-bound feeder cells, and/or at least one Fc-bound engineered particle (PM particle and/exosome) as detailed above. Fusion peptides can be provided in suitable containers along with other kit components such as cell reagents, cell growth media, selection media, protein purification reagents, buffers, and the like. The kits provided herein generally include instructions for carrying out the methods detailed below. Instructions included in the kits may be affixed to packaging material or may be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

### EXAMPLES

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. While the invention has been described with reference to particular embodiments and implementations, it will be understood that various changes and additional variations may be made and equivalents may be substituted for elements thereof without departing from the scope of the invention or the inventive concept thereof. In addition, many modifications may be made to adapt a particular situation or device to the teachings of the invention without departing from the essential scope thereof. Such equivalents are intended to be encompassed by the following claims. It is intended that the invention not be limited to the particular implementations disclosed herein, but that the invention will include all implementations falling within the scope of the appended claims.

### 1) Materials and Methods.

γδ T-cell expansion using CSTX-2-Fc feeder cells. Peripheral Blood Mononuclear Cells (PBMCs) were characterized for NK and T cell content and seeded at 200,000 NK cells per mL in RPMI1640 media supplemented with 10% fetal bovine serum, 1% antiobiotic-antimycotic and 100 U/mL hIL-2 (Peprotec). Cells were cocultured with irradiated or Mitomycin C-treated (50 μg/mL for 30 minutes) CSTX-002 or CSTX-002-Fc feeder cells that were added to the culture on day 0 at 1×10⁶ feeder cells per mL and day 7 at 500,000 feeder cells per mL. Cells were counted every other day and maintained at a concentration of 250,000 NK cells per mL. In experiments testing the effect of NK cells on γδ T-cell cells, PBMCs or CD56-depleted PBMCs were seeded at 1×10⁶ total cells per mL on day 0 and maintained at 250,000 total cells per mL every other day following re-stimulation with feeder cells on day 7. For experiments utilizing TCRα/β depleted PBMCs as starting material, cultures were seeded at 70,000 TCR γ/δ+ T-cells per mL on day 0 and maintained at 250,000 NK cells per mL every two days following re-stimulation with feeder cells on day 7.

Flow cytometry. For cell surface phenotyping, 50,000-100,000 cells were stained for 25 minutes at 4° C. with fluorescently labeled antibodies in 50 μL of flow cytometry buffer containing 0.5% Bovine Serum Albumin (BSA)+2 mM EDTA in Dubbelco's Phosphate Buffered Saline (DPBS). Samples were washed with flow buffer prior to analyzing on CytoFlex (Beckman Coulter) flow cytometer. The following pre-conjugated antibodies were used for detection: CD3-PacBlue (clone UCHT1), CD8a-PE-Cy7 (clone RPA-T8, CD56-PE (clone 5.1H11). TCR Vδ2 -APC-Fire750 (clone B6) and TCRαβ-APC (clone IP26) purchased from Biolegend and TCR Vδ1-FITC (clone REAL277) from Miltenyi Biotech.

### 2) Results.

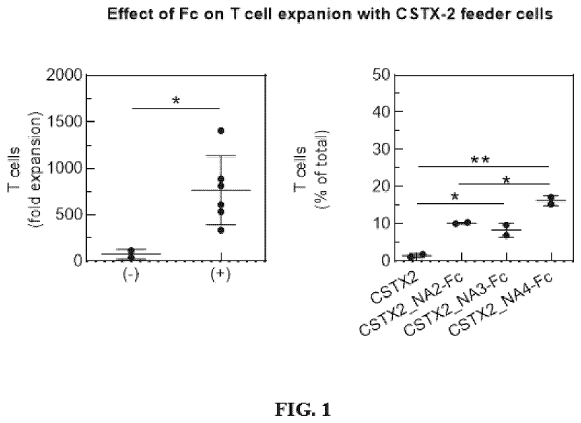

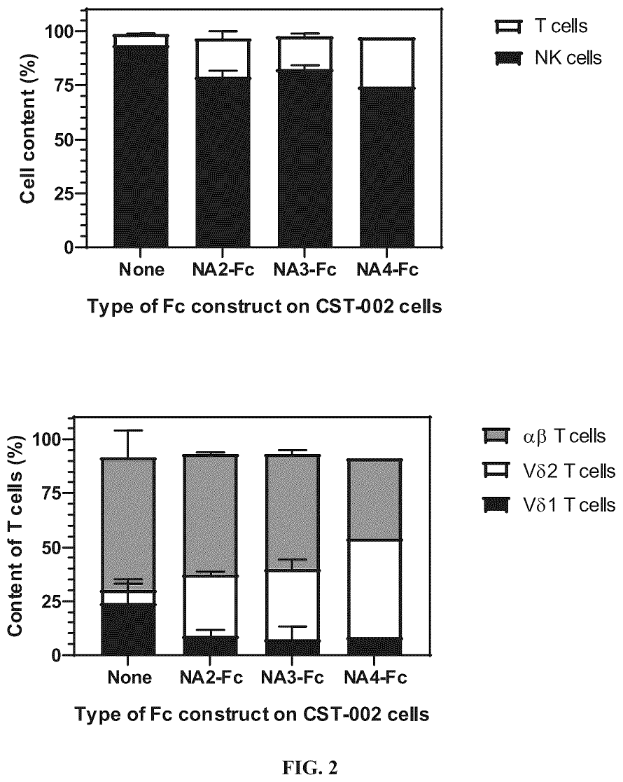

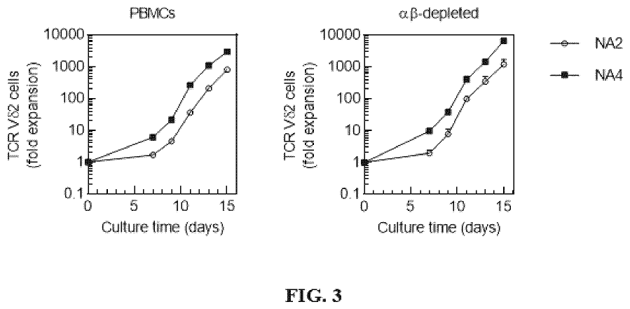

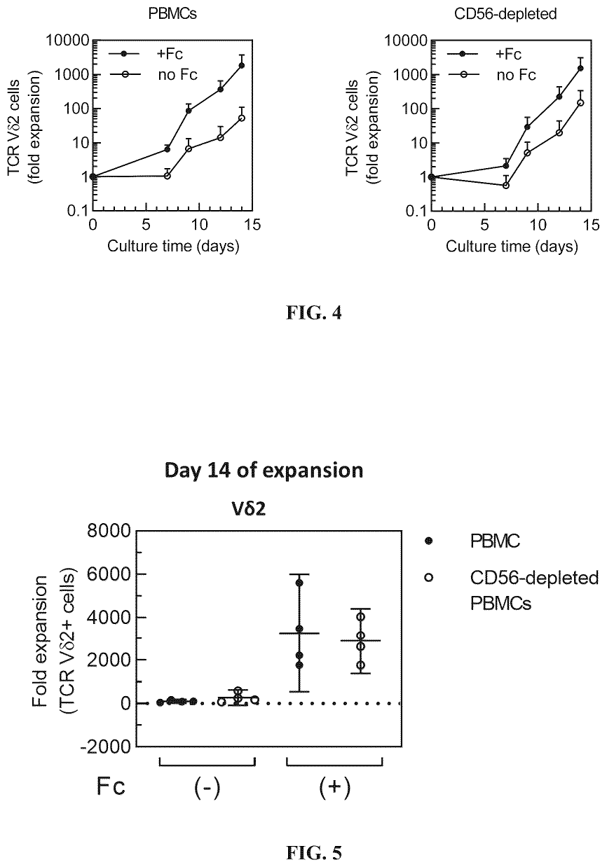

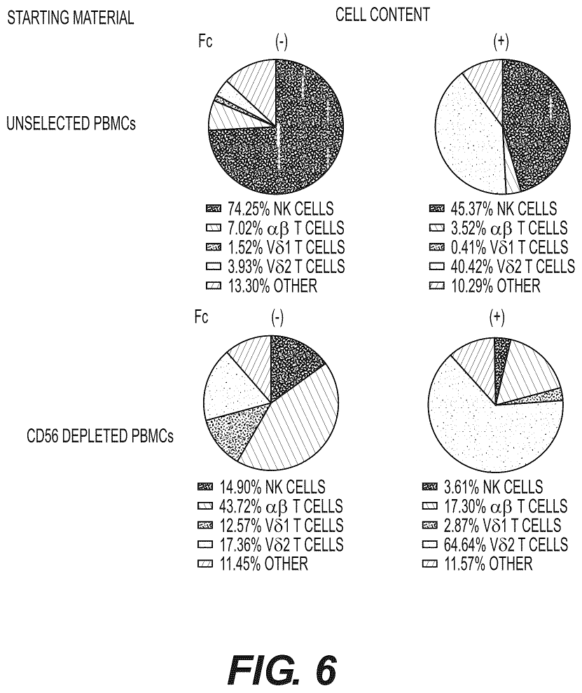

Gamma-delta (γδ) T-cells possess surface expression of CD16 (FcγRIIIa), the low-affinity receptor for IgG and can be responsive to stimulation with Fc of IgG1. To mimic antibody opsonized target cells, the K562 cell line containing other T and NK stimulating factors (membrane bound IL-21, 41BBL), and termed CSTX-002 cells were transduced to express Fc domain of IgG1 anchored to the surface via neuraminidase (NA) domain (FIG. 7). These CSTX-002-Fc cells were used in coculture with healthy donor PBMCs to stimulate γδ T-cells via CD16 engagement. Since antibody/ligand interaction results in large molecular complexes that can lead to steric effect playing role in the CD16/Fc interaction, the effect of length of the NA stalk on γδ T-cell expansion was tested first. PBMCs were stimulated with CSTX-002 cells or CSTX-002 cells expressing Fc fused to NA of varying lengths with NA2 being the shortest and NA4 being the longest (FIG. 8). The inclusion of Fc on the surface of CSTX-002 feeder cells resulted in an increase in T-cell content of co-cultured PBMC cells on day 14 (1.5% for CSTX-002 vs 16.3% for CSTX2-002_NA2-4-Fc; FIG. 1 left panel) with highest T cell content in cultures stimulated with Fc anchored to cell surfaced by the longest NA-stalk (NA4) (FIG. 1 right panel). Characterization of T-cell content revealed decrease in αβ T cells and increase of γδ T cells particularly Vγ9 Vδ2 subtype in cultures stimulated with Fc-expressing CSTX-002 cells as compared to CSTX-002 control (FIG. 2). The content of Vγ9 Vδ2 T-cells was the highest in cultures stimulated with Fc fused to NA stalk of the longest length (NA4-Fc). To test if elimination of other T-cell subpopulations that compete for nutrients and other factors can further improve expansion of γδ T-cells, αβ T-cells were depleted prior to co-culture with feeder cells and again the effect of neuraminidase stalk lengths (NA2 vs NA4) on expansion was compared. Depletion of αβ T-cells resulted in higher fold expansion of Vδ2 T-cells (PBMC 2990-fold vs αβ-depleted 6460-fold with NA4-Fc and PBMC 810-fold vs. αβ-depleted 1200 for NA2-Fc) (FIG. 3). Regardless of the presence of αβ T-cells, the increased length in NA resulted in higher fold expansion of Vδ2 T-cells. To test whether the Vδ2 T-cell expansion was dependent on another CD16 expressing cell type, NK cells in culture, CD56⁺ cells were depleted prior to co-culture with feeder cells and γδ T-cells expansion was monitored and compared to cultures utilizing untouched PBMCs. In both whole PBMC and CD56-depleted co-cultures, exposure to CSTX-002-Fc cells led to higher level of expansion of Vδ2 T-cells as compared to CSTX-002 control (FIG. 4). Similar level of expansion was observed in both untouched and CD56-depleted PBMC cultures with Vδ2 T-cells expansion >2,500-fold when co-cultured with CSTX-002-Fc compared to <300-fold when co-cultured with CSTX2 indicating that the expansion does not depend on the presence of NK cells in the starting material (FIG. 5). Furthermore, depletion of CD56-expressing cell populations from the starting material led to increased preferential expansion of Vδ2 T-cells and higher purity of the end-product when stimulated with Fc-CSTX-2 cells. Day 14 Vδ2 T-cells content in Fc-stimulated cultures was increased from 40% to 65% when CD56-depleted PBMCs were used instead of untouched PBMCs as starting material (FIG. 6). Stimulation with Fc led to preferential expansion of Vδ2 T-cells where Vδ2 T-cells comprised 64% of cells in CD56-depleted PBMC cultures exposed to CSTX-002-Fc after 14-days compared to 17% when co-cultured with CSTX-002 control cells lacking Fc (FIG. 6). The increase in the Vδ2 T-cells coincided with decrease in αβ T-cell population which was reduced from 44% in CSTX-002 control cultures to 17% in CSTX-002-Fc at day 14 when CD56-depleted PBMCs were used as a starting material. These results clearly support the use of CSTX2-Fc feeder cells for ex vivo expansion of γδ T-cells and indicate that longer lengths of the NA-anchor as well as use of γδ T-cell enriched starting material can positively impact the overall level of expansion.

1. A method for inducing, activating, and/or expanding of γδ T cells, comprising contacting at least one γδ T cell with an engineered feeder cell, an engineered plasma membrane particle, an exosome, or a solid support comprising a Fc domain bound to the external surface thereof.

2. The method of claim 1, wherein the Fc domain is bound to the external surface through a transmembrane domain.

3. The method of claim 2, wherein the transmembrane domain comprises a signal-anchor sequence selected from a transmembrane domain of neuraminidase, a signal-anchor sequence from parainfluenza virus hemagglutinin-neuraminidase, a signal-anchor sequence from the transferrin receptor, a signal-anchor sequence from the MHC class II invariant chain, a signal-anchor sequence from P glycoprotein, a signal-anchor sequence from asialoglycoprotein receptor, and a signal-anchor sequence from a neutral endopeptidase.

4. The method of claim 2, wherein the transmembrane domain comprises a parainfluenza virus hemagglutinin-neuraminidase (NA) peptide sequence.

5. The method of claim 4, wherein the NA peptide sequence comprises a sequence at least 81% identical to SEQ ID NO: 1, SEQ ID NO: 2, SEQ ID NO: 3, or SEQ ID NO: 4.

6. The method of claim 4, wherein the NA peptide sequence comprises a sequence at least 95% identical to SEQ ID NO: 1, SEQ ID NO: 2, SEQ ID NO: 3, or SEQ ID NO: 4.

7. The method of any one of claims 2 to 6, wherein the transmembrane domain and the Fc domain are linked via a peptide linker.

8. The method of any one of claims 1 to 7, wherein the Fc domain comprises an immunoglobulin Fc domain selected from IgG1, IgG2, IgG3, IgG4, IgA and IgE.

9. The method of any one of claims 1 to 8, wherein the Fc domain binds to CD16.

10. The method of any one of claims 1 to 9, wherein the feeder cell comprises a peripheral blood mononuclear cell (PBMC), a fibroblast, an epithelial cell, an endothelial cell, an antigen-presenting cell, or a microbial cell.

11. The method of any one of claims 1 to 9, wherein the feeder cell comprises RPMI8866, HFWT, 721.221, K562, or EBV-LCL.

12. The method of any one of claims 1 to 11, further comprising contacting the at least one γδ T cell with at least one γδ T cell effector agent.

13. The method of claim 12, wherein the at least one γδ T cell effector agent is expressed on or bound to the external surface of the engineered feeder cell, the engineered plasma membrane particle, the exosome, or the solid support.

14. The method of claim 12 or 13, wherein the at least one γδ T cell effector agent comprises a cytokine, an adhesion molecule, or a γδ T cell activating agent.

15. The method of any one of claims 12 to 14, wherein the at least one γδ T cell effector agent comprises 4-1BBL, CD80, CD86, MICA, UBLP, 2B4, LFA-1, ligand for NKG2D, ligand for DNAM-1, IL-2, IL-12, IL-18, IL-15, or IL-21, or any combination thereof.

16. The method of any one of claims 12 to [symbol] , wherein the at least one γδ T cell effector agent comprises 4-1BBL, IL-18, IL-15, or IL-21, or any combination thereof.

17. The method of any one of claims 1 to [symbol] , wherein the at least one γδ T cell is contacted with the feeder cell, the engineered particle, the exosome, or the solid support in vitro, in vivo, or ex vivo.

18. The method of any one of claims 1 to 17, wherein the expanded γδ T cells comprise Vδ2 subtype and/or Vδ1 subtype.

19. The method of claim any of claims 1-18, wherein the γδ T cells are autologous, haploidentical, or allogeneic γδ T cells.

20. The method of any of claims 1-19, wherein the γδ T cells are expanded for at least 14 days.

21. The method of claim 20, wherein at least about 5%, 10%, 20%, 30%, 40%, 50%, or 60% of the cells in the expanded cells are γδ T-cells of the Vδ2 subtype.

22. The method of any of claims 1-21, wherein the γδ T cells expand at a faster rate over 14 days than a control γδ T cell population.

23. The method of any of claims 1-22, wherein the γδ T cells are in a mixed cell population.

24. The method of claim 23, wherein the mixed cell population comprises NK cells.

25. The method of claim 24, wherein the mixed cell population is depleted of NK cells prior to, during, or after expansion of the γδ T cells.

26. A method of treating, decreasing, inhibiting, reducing, ameliorating, and/or preventing a cancer, metastasis, or an infectious disease in a subject comprising administering to the subject a therapeutically effective amount of γδ T cells expanded, activated, or induced according to the method of any one of claims 1-25.

27. A method of treating, decreasing, inhibiting, reducing, ameliorating, and/or preventing a cancer, metastasis, or an infectious disease in a subject comprising
a. obtaining at least one γδ T cell;
b. contacting the least one γδ T cell with an engineered feeder cell, an engineered plasma membrane particle, an exosome, or a solid support comprising a Fc domain bound to the external surface thereof;
c. administering to the subject a therapeutically effective amount of the contacted γδ T cells to the subject.

28. The method of claim 27, wherein the at least one γδ T cell is contacted with the feeder cell, the engineered particle, the exosome, or the solid support in vitro, in vivo, or ex vivo.

29. The method of claim 27, wherein step b further comprises inducing, activating, and/or expanding the at least one γδ T cell following the contact with the engineered feeder cell, the engineered plasma membrane particle, the exosome, or the solid support comprising a Fc domain bound to the external surface thereof.

30. The method of claim 29, wherein the γδ T cells are induced, activated, and/or expanded for at least 7 days.

31. A method of treating, decreasing, inhibiting, reducing, ameliorating, and/or preventing a cancer, metastasis, or an infectious disease in a subject by expanding, inducing, and/or activating endogenous γδ T cells in the subject, said method comprising administering to the subject an engineered plasma membrane particle, an exosome, or a solid support comprising a Fc domain bound to the external surface thereof.

32. The method of claims 27 to 31, wherein the Fc domain is bound to the external surface through a transmembrane domain.

33. The method of claim 32, wherein the transmembrane domain comprises a signal-anchor sequence selected from a transmembrane domain of neuraminidase, a signal-anchor sequence from parainfluenza virus hemagglutinin-neuraminidase, a signal-anchor sequence from the transferrin receptor, a signal-anchor sequence from the MHC class II invariant chain, a signal-anchor sequence from P glycoprotein, a signal-anchor sequence from asialoglycoprotein receptor, and a signal-anchor sequence from a neutral endopeptidase.

34. The method of claim 32, wherein the transmembrane domain comprises a parainfluenza virus hemagglutinin-neuraminidase (NA) peptide sequence.

35. The method of claim 34, wherein the NA peptide sequence comprises a sequence at least 81% identical to SEQ ID NO: 1, SEQ ID NO: 2, SEQ ID NO: 3, or SEQ ID NO: 4.

36. The method of claim 34, wherein the NA peptide sequence comprises a sequence at least 95% identical to SEQ ID NO: 1, SEQ ID NO: 2, SEQ ID NO: 3, or SEQ ID NO: 4.

37. The method of any one of claims 33 to 36, further comprising a peptide linker between the transmembrane domain and the Fc domain.

38. The method of any one of claims 31 to 37, wherein the Fc domain comprises an immunoglobulin Fc domain selected from IgG1, IgG2, IgG3, IgG4, IgA and IgE.

39. The method of any one of claims 31 to 38, wherein the Fc domain binds to CD16. (Original) The method of any one of claims 31 to 39, wherein the feeder cell comprises a peripheral blood mononuclear cell (PBMC), a fibroblast, an epithelial cell, an endothelial cell, an antigen-presenting cell, or a microbial cell.

41. The method of any one of claims 31 to 39, wherein the feeder cell comprises RPMI8866, HFWT, 721.221, K562, or EBV-LCL.

42. The method of any one of claims 31 to 41, further comprising contacting the γδ T cells with a least one γδ T cell effector agent.

43. The method of claim 42, wherein the γδ T cells are contacted with the at least one γδ T cell effector agent in vivo after administration of the γδ T cells to the subject.

44. The method of claim 42, wherein the γδ T cell effector agent is expressed on or bound to the membrane surface of the engineered feeder cell, the engineered plasma membrane particle, the exosome, or the solid support.

45. The method of any one of claims 10 to 44, further comprising administering to the subject an ex vivo composition comprising a fusion protein comprising a transmembrane domain linked to the amino terminus of an Fc domain and bound to an engineered feeder cell, an engineered plasma membrane particle, an exosome, or a solid support, in contact with an isolated mixed cell population comprising at least one γδ T cells comprising CD16 or a functional fragment thereof.

46. The method of claim 45, wherein the ex vivo composition is free of feeder cells and comprises an engineered plasma membrane particle or engineered exosome comprising an inverted Fc domain bound to an external surface of the engineered plasma membrane particle or exosome.

47. The method of claim 45 or 46, wherein the ex vivo composition further comprises at least one γδ T cell effector agent.

48. The method of any one of claims 45 to 47, wherein the engineered plasma membrane particle comprising a plasma membrane and a plurality of microparticles or support surfaces, wherein the plasma membrane coats the plurality of microparticles or support surfaces.

49. The method of claim 48, wherein the plurality of microparticles or surfaces comprise at least one of magnetic microparticles, silica beads, polystyrene beads, latex beads, micro-structures, a contrast agent, and a cancer therapeutic agent.

50. The method of claim any of claims 42 to 49, wherein the at least one γδ T cell effector agent comprises a cytokine, an adhesion molecule, a γδ T cell activating agent, or a γδ T cell inhibitor agent.

51. The method of any of claims 42 to 50, wherein the at least one γδ T cell effector agent comprises 4-1BBL, CD80, CD86, MICA, UBLP, 2B4, LFA-1, ligand for NKG2D, ligand for DNAM-1, IL-2, IL-12, IL-18, IL-15, or IL-21.

52. The method of any of claims 42 to 51, wherein the at least one γδ T cell effector agent comprises 4-1BBL, IL-18, IL-15, or IL-21, or any combination thereof.

53. The method of claim and of claims 31 to 51, wherein the γδ T cells are autologous, haploidentical, or allogeneic γδ T cells.

54. The method of any of claims 31 to 53, wherein the cancer is selected from the group consisting of a hematologic cancer, lymphoma, colorectal cancer, colon cancer, lung cancer, a head and neck cancer, ovarian cancer, prostate cancer, testicular cancer, renal cancer, skin cancer, cervical cancer, pancreatic cancer, and breast cancer. In one aspect, the cancer comprises a solid tumor. In another aspect, the cancer is selected from acute myeloid leukemia, myelodysplastic syndrome, chronic myeloid leukemia, acute lymphoblastic leukemia, myelofibrosis, multiple myeloma. In another aspect, the cancer is selected from a leukemia, a lymphoma, a sarcoma, a carcinoma and may originate in the marrow, brain, lung, breast, pancreas, liver, head and neck, skin, reproductive tract, prostate, colon, liver, kidney, intraperitoneum, bone, joint, eye.

55. The method of claim 54, further comprising administering to the subject at least one cancer therapeutic agent in combination with the composition.

56. The method of claim 55, wherein the at least one cancer therapeutic agent is selected from the group consisting of Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane),Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPDX, Carac (Fluorouracil--Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate).

57. The method of claim 55, wherein the at least one cancer therapeutic agent is selected from a chemotherapy agent (eg CHOP, FLAG, 7+3), a drug based preparative regimen, or a combination thereof. (Cy-Flu, Bu-Flu, Flu-Mel).

58. The method of any one of claims 31 to 57, wherein the infectious disease is caused by a viral infection, bacterial infection, fungal infection, or parasitic infection.

59. The method of claim 58, wherein the viral infection comprises an infection of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papillomavirus, Respiratory syncytial virus, Adenovirus, Coxsackie virus, Dengue virus, Mumps virus, Poliovirus, Rabies virus, Rous sarcoma virus, Reovirus, Yellow fever virus, Zika virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, or Human Immunodeficiency virus type-2.

60. The method of claim 58, wherein the bacterial infection comprises an infection of Mycobaterium tuberculosis, Mycobaterium bovis, Mycobaterium bovis strain BCG, BCG substrains, Mycobaterium avium, Mycobaterium intracellular, Mycobaterium africanum, Mycobaterium kansasii, Mycobaterium marinum, Mycobaterium ulcerans, Mycobaterium avium subspecies paratuberculosis, Nocardia asteroides, other Nocardia species, Legionella pneumophila, other Legionella species, Acetinobacter baumanii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii, other Bordetella species, Burkholderia mallei, Burkholderia psuedomallei, Burkholderia cepacian, Chlamydia pneumoniae, Chlamydia trachomatis, Chlamydia psittaci, Coxiella burnetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, Clostridium difficile, other Clostridium species, Yersinia enterolitica, and other Yersinia species, and Mycoplasma species.

61. The method of claim 58, wherein the fungal infection comprises an infection of Candida albicans, Cryptococcus neoformans, Histoplama capsulatum, Aspergillus fumigatus, Coccidiodes immitis, Paracoccidiodes brasiliensis, Blastomyces dermitidis, Pneumocystis carinii, Penicillium marneffi, or Alternaria alternate.

62. The method of claim 58, wherein the parasitic infection comprises an infection of Toxoplasma gondii, Plasmodium falciparum, Plasmodium vivax, Plasmodium malariae, other Plasmodium species, Entamoeba histolytica, Naegleria fowleri, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Diphyllobothrium latum, Clonorchis sinensis; Clonorchis viverrini, Fasciola hepatica, Fasciola gigantica, Dicrocoelium dendriticum, Fasciolopsis buski, Metagonimus yokogawai, Opisthorchis viverrini, Opisthorchis felineus, Clonorchis sinensis, Trichomonas vaginalis, Acanthamoeba species, Schistosoma intercalatum, Schistosoma haematobium, Schistosoma japonicum, Schistosoma mansoni, other Schistosoma species, Trichobilharzia regenti, Trichinella spiralis, Trichinella britovi, Trichinella nelsoni, Trichinella nativa, or Entamoeba histolytica.

63. The method of any one of claims 31 to 62, wherein the γδ T cells are formulated in a pharmaceutically acceptable carrier and a pharmaceutically acceptable excipient.

64. The method of any one of claims 31 to 63, wherein the γδ T cells are administered parenterally, intravenously, intraperitoneally, or subcutaneously, or through arterial infusion, venous infusion, or artificial catheter mediated infusion.
